(19) United States
(12) Patent Application Publication
(10) Pub. No.: US 2026/0234564 A1
(43) Pub. Date: Aug. 13, 2026

(54) CELL POPULATIONS IN THE ANORECTAL TRANSITION ZONE WITH TISSUE REGENERATIVE CAPACITY, AND METHODS FOR ISOLATION AND USE THEREOF

(71) Applicant: Curileum Discovery Ltd, Harrow (GB)

(72) Inventors: Jeffrey G. Moore, London (GB); Priti Kalra, London (GB)

(21) Appl. No.: 19/156,554

(22) PCT Filed: Feb. 15, 2024
(86) PCT No.: PCT/GB2024/050415

§ 371 Date: Aug. 14, 2025

Related U.S. Application Data
(60) Provisional application No. 63/485,521, filed on Feb. 16, 2023.

(51) Int. Cl. C12N 5/071; A61K 35/38; A61P 1/16

(52) U.S. Cl. CPC C12N 5/068; A61K 35/38; A61P 1/16; C12N 5/067; C12N 5/0676; C12N 5/0688; C12N 2506/23

(57) ABSTRACT

Provided herein are compositions of anorectal transition zone stem cells, including multipotent and progenitor cells, to treat endodermal disease. Also provided are cellular compositions of the anorectal transition zone stem cells, including multipotent and progenitor cells, in a pharmaceutically acceptable carrier.

### BACKGROUND

Transition zones of the gastrointestinal tissues exist between two different epithelial tissue types. Anorectal transition zone tissue is at the junction of rectal columnar epithelium of endodermal embryological origin and the anal stratified squamous, non-keratinised epithelium of ectodermal embryological origin (McNairn and Guasch (2011), “Epithelial transition zones: merging microenvironments, niches, and cellular transformation,” Eur J Dermatol, 21 (Suppl 2): 21-8). Histopathological labeling studies of mouse and human anorectal transition zone tissue have identified cells expressing p63, a marker for basal cells; cytokeratin 7 (CK7), a marker for simple columnar cells (Yang et al. (2015), “Microanatomy of the cervical and anorectal squamocolumnar junctions: a proposed model for anatomical differences in HPV related cancer risk,” Mod Pathol, 28(7): 994-1000); and CD34, a marker for stem cells and progenitor cells (McNairn, supra). Further studies in mice have supported the presence of stem cells and progenitor cells in the anorectal transition zone using a label-retaining cell assay and immunohistochemistry confirming expression of p63, CK7, CD34, and identifying expression of SOX2, a pluripotent stem cell marker (Runck et al. (2010), “Identification of epithelial label-retaining cells at the transition between the anal canal and the rectum in mice,” Cell Cycle, 9(15): 3039-45).

Stem cell therapy offers promise to repair endodermal-derived tissues and organs that are dysfunctional or damaged by infection, medications, or physical trauma. Each of the current cell therapy sources have limitations in their utilities.

Human embryonic stem cells (ESCs) are pluripotent, giving rise to all cells in the body. They have regenerative potential but pose risks for teratoma formation and eliciting autoimmune responses. Encapsulating ESCs mitigates some of these risks. iPSCs, fibroblasts reprogrammed to differentiated cells, have mutagenic potential due to reprogramming methods, and have barriers to long-term transplant viability and functionality. Mesenchymal stem cells (MSCs) are widely used for their inherent anti-inflammatory properties, which reduce the risks of autoimmune responses and tumourigenicity. However, since MSCs are not regenerative, their benefit is transitory.

### SUMMARY

The invention is based, in part, upon the discovery of compositions for use (or suitable for use) in treating a tissue or organ derived from the endoderm in a subject in need thereof, methods of making stem cell compositions useful in the treatment of an endodermal tissue or organ, and methods of treating an endodermal tissue or organ with such a stem cell composition.

In one aspect, provided herein are stem cell compositions for treating an endodermal tissue or organ, the composition comprising isolated anorectal transition zone (ATZ) cells and/or ATZ-derived organoid cells. The isolated ATZ cells preferably include isolated ATZ crypt cells, including ATZ multipotent stem cells. The ATZ-derived organoid cells preferably include ATZ multipotent stem cells and ATZ-derived endoderm cells which can be single cells, clusters of cells, 2-dimensional layers of cells, or 3-dimensional organoids. The cells can be cultured in suspension, adherent layers or liquid cultures. The compositions may comprise multipotent ATZ stem cells and endodermal progenitor ATZ cells.

The isolated ATZ crypt cells comprise multipotent stem cells and/or progenitor cells that express KIT, LGR5, NANOG, and OCT4A. The multipotent isolated ATZ crypt cells can also express one or more additional stem cell and progenitor cell markers selected from the group consisting of BMP4, CD34, CXCR4, and SOX17. In some embodiments, the multipotent ATZ crypt cells express at least two, three or four stem cell and progenitor cell markers selected from the group consisting of BMP4, CD34, CXCR4, and SOX17.

The ATZ-derived organoids can express additional endoderm markers depending on the specific endodermal lineage-specific medium (LSM) used.

For example, in some embodiments, the ATZ-derived crypt cells are grown in a pluripotent stem cell MGM (e.g., mTesR™, STEMCELL Technologies, Inc, Vancouver, CA; StemFit® Feeder-Free Stem Cell Culture Media, AMSBIO, Abingdon, UK).

For example, in some embodiments, the ATZ-derived crypt cells are grown in a definitive endoderm LSM (e.g., Gibco™ PSC Definitive Endoderm Induction Kit, Thermo Fisher Scientific, Waltham, Massachusetts).

The isolated ATZ crypt cells or ATZ-derived organoids do not express CD45 and, therefore, are not of hematopoietic lineage.

In some embodiments, the ATZ-derived crypt cells grown in an intestinal-specific LSM (e.g., IntestiCult™; Human Intestinal Organoid Culture Protocol, Bio-Techne, Minneapolis, Minnesota) can, in addition, express ALP.

In some embodiments, the ATZ-derived crypt cells grown in a pancreas-specific LSM (e.g., PancreaCult™; Human Pancreas Organoid Culture Protocol, Bio-Techne) can express one or more additional markers selected from the group consisting of AMY, CK19, FOXA2, GCG, INS, NKX6.1, and SOX9. In some embodiments, ATZ-derived crypt cells grown in a pancreas-specific LSM can express two, three, four, five, six or seven additional markers selected from the group consisting of AMY, CK19, FOXA2, GCG, INS, NKX6.1, and SOX9.

In some embodiments, the ATZ-derived crypt cells grown in liver-specific LSM (e.g., HepatiCult™; Human Liver Organoid Culture Protocol, Bio-Techne) can express one or more of the additional markers from the group consisting of AFP, ALB, CK19, EPCAM, FOXA2, HNF4A, and SOX9. In some embodiments, ATZ-derived crypt cells grown in a liver-specific LSM can express two, three, four, five, six or seven additional markers selected from the group consisting of AFP, ALB, CK19, EPCAM, FOXA2, HNF4A, and SOX9.

In some embodiments, the ATZ-derived crypt cells grown in lung-specific LSM (e.g., STEMdiff™ Branching Lung Organoid Kit, or PneumaCult™ Airway Organoid Kit, or PneumaCult™ Apical-Out Airway Organoid Medium, or PneumaCult™ Alveolar Organoid Medium, all from STEMCELL Technologies; Human Lung Organoid Culture Protocols, Bio-Techne; 3dGRO™ Lung Organoid Branching Medium3dGRO™ and Lung Organoid Maturation Medium, Merck) can express one or more of the additional markers from the group consisting of SOX9, NKX2.1, TMPRSS2, MUC1, VIM, and ACE2. In some embodiments, ATZ-derived crypt cells grown in a lung-specific LSM can express two, three, four, five or six additional markers selected from the group consisting of SOX9, NKX2.1, TMPRSS2, MUC1, VIM, and ACE2.

ATZ-derived crypt cells grown in thyroid-specific LSM (as directed in the protocol in “Adult mouse and human organoids derived from thyroid follicular cells and modelling of Graves' hyperthyroidism”, PNAS, 118(51): e1-11)) can express one or more of the additional markers from the group consisting of as PAX8 and NKX2.1.

The isolated ATZ crypt cells or ATZ-derived organoids can be combined with an exogenous biocompatible scaffold, for example, a synthetic scaffold or a biological scaffold. Depending upon the circumstances, the scaffold can be collagen-based. Alternatively, the isolated ATZ crypt cells or ATZ-derived organoid cells can be combined with a matrix material (e.g., a gel) or a pharmaceutically acceptable carrier (e.g., saline).

The isolated ATZ crypt cells or ATZ-derived organoids can be porcine cells or human cells.

The isolated ATZ crypt cells or ATZ-derived organoids can be allogeneic or autologous to the subject in need of treatment.

The isolated ATZ crypt cells or ATZ-derived organoid can be genetically-modified to include selectable markers (e.g., to detect engraftment), to eliminate the cells (e.g., a suicide gene) or to reduce a host-versus-graft response (e.g., MHC or B2M knock-outs).

In certain embodiments, the composition is cryopreserved. To facilitate cryopreservation, the composition may comprise a suitable cryopreservation media.

In another aspect, the invention provides methods of preparing the pharmaceutical compositions (e.g., any of the cellular compositions as disclosed herein). The methods comprise (a) harvesting ATZ tissue from a donor (e.g., a porcine donor or a human donor); (b) enzymatically digesting the tissue to prepare a cell suspension; (c) optionally combining at least a portion of the cell suspension with media for cryopreservation media and cryopreserving the cell suspension; and (d) combining the cell suspension of step (b) or the thawed suspension of optional step (c) with an exogenous biocompatible scaffold, matrix, or pharmaceutically acceptable carrier.

The cell suspension comprises isolated ATZ crypt cells and/or ATZ-derived organoid cells, which include multipotent stem cells. The ATZ crypt cells and/or ATZ-derived organoid cells, can also include endoderm progenitor cells. In certain embodiments, the ATZ cells can differentiate into endodermal cells (e.g., that express SOX17).

In some embodiments, the ATZ stem cells express CD34, CD117 (KIT), and CD184 (CXCR4), for example, as detected by flow cytometry. In addition, the ATZ stem cells do not express detectable levels of CD45, for example, as detected by flow cytometry. Furthermore, NANOG and/or OCT4A can be detected on freshly isolated ATZ crypt cells or ATZ-derived organoids.

In certain embodiments, the cells in the suspension (e.g., ATZ stem cells) are expanded in vitro.

In some embodiments, in step (d) of the method described above, cells in the cell suspension are combined with an exogenous biocompatible scaffold, for example, a synthetic scaffold or a biological scaffold. In certain embodiments, the scaffold comprises a collagen-based scaffold. Alternatively, the isolated ATZ crypt cells or ATZ-derived organoid cells can be combined with a matrix material (e.g., a gel) or a pharmaceutically acceptable carrier (e.g., saline).

In another aspect, the invention provides cellular compositions produced by the methods disclosed herein.

In another aspect, the invention provides methods for treating damaged or dysfunctional gastrointestinal mucosa tissue (e.g., a gastric mucosa or intestinal mucosa) in a subject in need thereof, the methods comprising administering into the damaged or dysfunctional gastrointestinal mucosa tissue an effective amount of a cellular composition disclosed herein, thereby to treat the damaged or dysfunctional gastrointestinal mucosa tissue.

In another aspect, the invention provides methods for treating damaged or dysfunctional pancreas tissue in a subject in need thereof, the methods comprising administering into the damaged or dysfunctional pancreas tissue an effective amount of a cellular composition disclosed herein, thereby to treat the damaged or dysfunctional pancreas tissue.

In another aspect, the invention provides methods for treating damaged or dysfunctional liver tissue in a subject in need thereof, the methods comprising administering into the damaged or dysfunctional liver tissue an effective amount of a cellular composition disclosed herein, thereby to treat the damaged or dysfunctional liver tissue.

In another aspect, the invention provides methods for treating damaged or dysfunctional lung tissue in a subject in need thereof, the methods comprising administering into the damaged or dysfunctional lung tissue an effective amount of a cellular composition disclosed herein, thereby to treat the damaged or dysfunctional lung tissue.

### BRIEF DESCRIPTION OF THE DRAWINGS

FIG. 1 shows the isolation of ATZ crypts from porcine tissue: (a) shows resected colorectum tissue; (b) shows exposed mucosal epithelium, (c) shows excised dentate line, (d) shows freshly isolated ATZ crypts, (e) shows freshly isolated ATZ crypts, (f) shows freshly isolated ATZ crypts, (g) shows freshly isolated bifurcating ATZ crypt, (h) shows freshly isolated ATZ submucosal gland, and (i) shows freshly isolated ATZ submucosal gland.

FIG. 2 shows the isolation of ATZ crypts from Crohn's disease patient tissue: (a) shows resected anorectal tissue, (b) shows excised mucosal epithelium, (c) shows brightfield images of freshly isolated ATZ crypts, and (d) shows brightfield images of freshly isolated ATZ crypts.

FIG. 3 shows (a) crypt organoid development in porcine GI tissue and (b) crypt organoid development in Crohn's disease patient rectum and ATZ tissue.

FIG. 4 shows the increased progenitor capacity of single cell preparations of porcine ATZ crypts compared to rectal crypts. FIG. 4(a) shows porcine ATZ and rectal crypt growth over 4 weeks. FIG. 4(b) shows increased plating efficiencies over two months of crypt organoids derived from ATZ crypt-derived cells compared to rectal crypt-derived cells. Cells were isolated and replated after 1 month.

FIG. 5 shows protein expression of stem cell and progenitor cell markers on freshly isolated crypts and crypt organoids derived from pigs and a Crohn's disease patient: (a) shows CD117 expression on crypt-derived single cells from porcine GI tissues, (b) shows CD34 expression on porcine fresh crypt-derived single cells from porcine GI tissues, (c) shows double labeling by flow cytometry of CD34 and CD117 expression on fresh porcine ATZ crypt cells, (d) shows stem cell and progenitor marker expression on porcine crypt organoids (small intestine is shown on the left bar, rectum is shown in the middle bar, ATZ is shown on the right bar), (e) shows the indicated developmental lineage marker expression on fresh porcine ATZ crypt-derived cells, (f) shows stem cell and progenitor marker expression on Crohn's disease patient rectal crypt-derived organoids.

FIG. 6 shows mRNA profiling from freshly isolated crypts and crypt organoids of the porcine ATZ: (a) mRNA profiling of freshly isolated porcine ATZ crypt cells, (b) shows mRNA profiling of organoids derived from porcine ATZ crypt cells.

FIG. 7 shows flow cytometric analysis of stem cell and progenitor cell markers to assess growth media at day 14 to promote porcine ATZ crypt stem cell expansion, wherein (a) shows CD117 expression, (b) shows Brachyury expression, and (c) shows SOX17 expression.

FIG. 8 shows that porcine ATZ crypt cells generate mature cells of the endoderm lineage: (a)-(d) shows brightfield images of organoid development from days 1, 4, 7, and 12, respectively, (e)-(h) shows DAPI staining of nuclei and immunocytochemistry images of organoids, wherein, (e) shows lysosome expression, (f) shows Ki67 expression, (g) shows Muc-2 expression, (h) shows CK18 expression.

FIG. 9 shows flow cytometry analysis of endodermal markers on ATZ organoids at days 0, 7, and 14.

FIG. 10 shows that porcine ATZ crypt cells generate mature cells of the mesoderm lineage: (a)-(d) shows brightfield images of blood vessel-like development, wherein (a) shows undifferentiated ATZ single cells at day 0, (b) shows ATZ single cells starting to form clusters at day 3, (c) shows blood vessel-like structures starting to form at day 8, (d) shows a network of blood vessel-like structures forming at day 10. (e) shows DAPI stained nucleus and immunocytochemistry of CD31 expressing endothelial-like cells, and (f) shows DAPI stained nucleus and immunocytochemistry of CD31 expressing blood vessel-like cells.

FIG. 11 shows that porcine ATZ crypt cells generate mature cells of the mesoderm lineage as detected by flow cytometry for mesodermal markers, brachyury, and CD31, on ATZ organoids at days 0, 7, and 14.

FIG. 12 shows porcine ATZ crypt cells generate mature cells of the ectoderm lineage: (a) shows brightfield images of keratinocyte development from anal skin and ATZ crypts grown in KFSM, (b) shows immunocytochemistry of K14 and K15 markers on anal skin cultures, (c) shows immunocytochemistry of K14 and K15 markers on ATZ crypt cultures, (d) shows flow cytometry analysis of ectodermal markers PAX6, NESTIN, and CK14 on ATZ cultured cells on days 0, 7 and 14.

FIG. 13 shows brightfield images of porcine small intestine crypt single cells in medium to promote differentiation in the 3 developmental lineages at days 0, 7, and 14, wherein (a) shows the culture in human endodermal medium (IntestiCult™), (b) shows culture in human mesodermal medium (MethoCult™), (c) shows culture in ectodermal medium (KFSM).

FIG. 14 shows the results of an in vitro embryoid body assay to test pluripotency of porcine ATZ crypt single cells: (a) shows brightfield images of ATZ embryoid body development, (b) shows alkaline phosphatase staining of ATZ embryoid bodies, (c) shows immunostaining of ATZ embryoid bodies for expression of pluripotent stem cell markers SSEA4 and OCT4, (d) shows immunostaining of ATZ embryoid bodies with pluripotent stem cell markers SOX2 and TRA-1-60 expression. DAPI was used for nucleus staining.

FIG. 15 shows isolation of porcine pancreatic ducts: (a) resected pancreas; (b) minced pancreatic tissues after enzymatic digestion; (c) isolated pancreatic ducts and single cells.

FIG. 16 shows porcine pancreatic ductal organoid development derived from ATZ and pancreatic tissues grown in PancreaCult™. For ATZ: (a) clusters of cells formed on day 4, filled ring structures formed on day 7, and compact, thickened organoids formed on day 14. For pancreas: (b) cystic organoid structures formed on day and day 2 after passage; compact, thickened organoids formed on day 4 after passage. FIG. 16(c) shows a comparison of cystic organoids derived from ATZ crypts and pancreatic ducts. FIG. 16(d) shows a comparison of compact, thickened organoids derived from ATZ crypts and pancreatic ducts.

FIG. 17 shows protein markers detected by immunofluorescence of porcine ATZ crypt-derived organoids grown in PancreaCult™ at days 4 and 7: (a) cytokeratin 19 and (b) insulin.

FIG. 18 shows isolation of porcine liver stem cells: (a) resected liver; (b) minced liver tissues after enzymatic digestion; (c) isolated liver crypts and single cells.

FIG. 19 shows porcine liver crypt organoid development derived from ATZ and liver stem cells grown in HepatiCult™. For ATZ: (a) clusters of cells formed on day 4, filled ring structures formed on day 7, and compact, thickened organoids formed on day 14. For liver: b) small cystic organoid structures formed at day 3, larger cystic organoid structures at days 1 and 4 after passage.

FIG. 20 shows protein markers detected by immunofluorescence of porcine ATZ crypt-derived organoids in grown in HepatiCult™ at days 4 and 7: (a) cytokeratin 19, (b) albumin.

FIG. 21 shows the isolation of ATZ crypts from an idiopathic perianal fistula patient: (a) biopsy taken from anorectal tissue, (b) brightfield images of freshly isolated ATZ crypts, and (c) brightfield images of freshly isolated ATZ single cell suspension.

FIG. 22(a-b) shows mRNA expression by qPCR of freshly isolated ATZ crypts of two idiopathic fistula perianal patients. Ct values are normalized to housekeeping gene GAPDH.

FIG. 23 shows organoid development in IntestiCult™ from a single cell suspension generated from ATZ crypts of idiopathic perianal fistula patient. Clusters of cells formed on day 4 and organized structures formed on day 7 (both at passage 0); compact, thickened organoids formed on day 7 and day 14 of passage 1.

FIG. 24 shows formation of pancreatic organoids derived from idiopathic perianal fistula patient ATZ cells and cultured in PancreaCult™.

FIG. 25 shows an in vivo study of donor male porcine ATZ cells implanted into the pancreas of a recipient female pig: (a) a single cell suspension of male ATZ donor cells was thawed and mixed with a collagen paste, and (b) injected into 3 sites in the recipient pig pancreas. FIG. 25(c) shows a suture securing the white collagen paste. After 5 weeks, the animal was sacrificed. FIG. 25(d) shows the resected pancreas (arrows indicating implantation sites).

FIG. 26 shows histological staining of porcine pancreatic tissue recovered from the ATZ cell implantation study in EXAMPLE 28. The top panel shows picrosirius red staining (dark gray) of collagen fibers, consistent with newly formed matrix. The bottom panel shows fluorescence in situ hybridization (FISH) of the SRY gene, indicating the presence of male ATZ donor cells in the recipient pig pancreas.

FIG. 27 shows mRNA profiling by RNA Seq of male donor ATZ crypt cells cultured in IntestiCult™, cells harvested from the ATZ/Permacol™ implant in pancreas, and control pancreatic tissue harvested from the ATZ implantation study in EXAMPLE 28: (a) mRNA counts of NKX6-1, INS, MUC-2 genes, (b) differential gene expression of pancreas cell-type specific marker genes comparing cells in the ATZ/Permacol™ implant to ATZ cells cultured in IntestiCult™, (c) differential gene expression of pancreas cell-type specific marker genes in the ATZ/Permacol™ implant to control pancreatic tissue.

FIG. 28 shows formation of liver organoids derived from idiopathic patient ATZ crypt cells and cultured in HepatiCult™. A ring formed on day 4, larger cystic organoids at day 2 and day 6 after passage, and finally, filled in, compact, thickened organoids formed by day 14 of passage 1.

FIG. 29 shows an in vivo study of donor male porcine ATZ crypt cells implanted into the liver of a recipient female pig: (a) injection into 3 sites in the recipient pig liver. After 5 weeks, the animal was sacrificed, (b) the liver was resected and (c) sections were placed in cassettes and fixed. The implantation sites were still visible (indicated by arrows in the image) in the resected tissues.

FIG. 30 shows histological staining of porcine liver tissue recovered from the ATZ implantation study in EXAMPLE 32: (a) a section of liver with newly forming complex structures of large and small vasculature or ducts visualised by staining: haematoxylin and eosin (top left panel), collagen fibre staining detected by picrosirius red (dark grey) in the upper right panel, and albumin (light grey) in the lower left panel and in the insert in the lower right panel.

FIG. 30(b) a section of liver stained with haematoxylin and eosin and picrosirius red in the upper panels. Within the ATZ/Permacol™ implant, a cluster of albumin producing cells was detected in the lower left panel and in the insert in the lower right panel.

FIG. 30(c) shows a section of liver distal from the ATZ/Permacol™ implant site stained with haematoxylin and eosin identifying apparent regenerating liver tissue in the upper panel and highlighted in the region of selected interest in the lower panel.

FIG. 30(d) show staining by FISH for the SRY male gene (light grey) highlighted in the region of selected interest in the lower panel.

FIG. 31 shows mRNA profiling by RNA Seq of male donor ATZ crypt cells cultured in IntestiCult™, cells harvested from the ATZ/collagen implant in liver, and control liver tissue harvested from the ATZ implantation study in EXAMPLE 32: (a) Normalised mRNA count of SOX9, ALB, MUC-2 genes, (b) Differential gene expression of liver cell-type specific marker genes comparing cells harvested from the ATZ/Permacol™ in liver to ATZ cells cultured in IntestiCult™ (c) Differential gene expression of liver cell-type specific marker genes comparing cells harvested from the ATZ/Permacol™ in liver implant to control liver tissue.

### DETAILED DESCRIPTION

### Definitions

As used herein, the following terms and phrases shall have the meanings set forth below. Unless defined otherwise, all technical and scientific terms used herein have the same meaning as commonly understood by one of ordinary skill in the art to which this disclosure belongs.

As used herein, the term “gastrointestinal mucosa” or “gastrointestinal mucosal epithelium” refers to the epithelial mucosal layers of the gastrointestinal tract including the mouth, pharynx (throat), oesophagus, stomach, small intestine, large intestine, rectum, and anus. The “gastrointestinal mucosa” or “gastrointestinal mucosal epithelium” includes the oral, pharyngeal, oesophageal, gastric, intestinal, rectal and anal mucosa or mucosal epithelium.

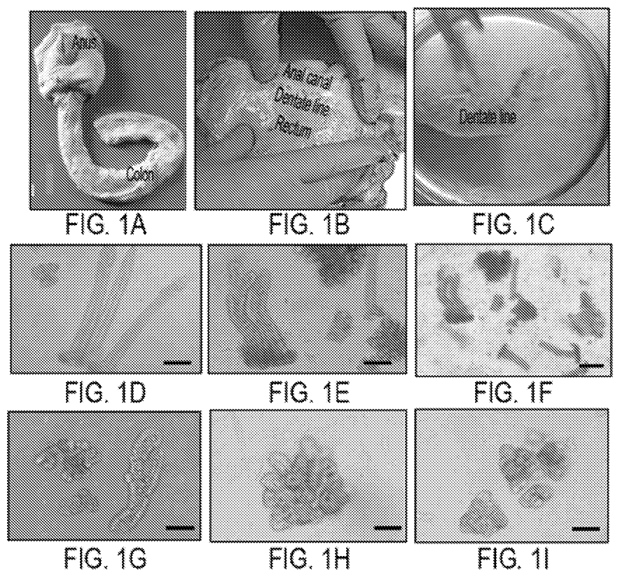

As used herein, the term “anorectal transition zone” or “ATZ” refers to the intestinal epithelium interposed between the uninterrupted squamous epithelium of the anoderm and dentate (or pectinate) line below and the uninterrupted rectal columnar epithelium above. The dentate line is the junction between the superior and inferior anal canal. There are many differences between these two regions, including their embryological origins, innervation, venous and arterial supply, and lymphatic supply. Above the dentate line, the epithelium of the anal canal has an endodermal origin and is lined by simple columnar epithelia. Below the dentate line, the epithelium of the anal canal has an ectodermal origin and is predominantly lined by stratified squamous epithelium. The epithelium of the ATZ is typically 1-4 mm wide and can easily be identified and biopsied by those skilled in the art (see, e.g., FIG. 1).

As used herein, the term “anorectal transition zone cells” or “ATZ cells” refers to a mixed population of cells derived from anorectal transition zone epithelial tissue comprising crypts, submucosal glands, and other epithelial cells.

As used herein, the term “isolated ATZ cells” refers to a mixed population of cells physically isolated (e.g., by biopsy sample) from the ATZ epithelium. Optionally, the ATZ tissue can be physically minced or enzymatically digested to isolate ATZ cells.

As used herein, the term “crypt cells” refers to the cells of the crypts of Lieberkühn, structures below the surface of the intestinal mucosal lining, and comprising stem cells that are responsible for continuously regenerating intestinal mucosa throughout life.

As used herein, “ATZ crypt cells” refers to the crypt cells of the ATZ, which include multipotent stem cells (i.e., cells that have the capacity to self-renew by dividing and to develop into multiple specialised cell types present in a specific tissue or organ), progenitor cells, and mature crypt cells. Multipotent ATZ cells are stem cells that have the capacity to differentiate into multiple somatic cell lineages including endodermal cells (e.g., intestinal mucosa), mesodermal cells (e.g., blood vessels), and/or ectodermal cells (e.g., skin).

As used herein, the term “isolated ATZ crypt cells” refers to a mixed population of cells (e.g., multipotent stem cells, progenitor cells, fully differentiated or mature cells) forming the crypts that have been physically isolated (e.g., by dissection) from the ATZ epithelium. Alternatively, or in addition, the ATZ crypts can be physically, chemically, or enzymatically dissociated to further isolate the ATZ crypt cells.

As used herein, the term “stem cells” refers to undifferentiated or partially differentiated cells that can differentiate into various types of cells and proliferate indefinitely to produce more of the same stem cell (i.e., self-renewing).

As used herein, “self-renewal” is the process by which stem cells divide to make more stem cells, perpetuating the stem cell pool throughout life. Self-renewal is division with maintenance of the undifferentiated state. This requires cell cycle control and often maintenance of multipotency or pluripotency, depending on the stem cell.

As used herein, the term “progenitor cells” refers to stem cells with the potential to differentiate into a single cell type or lineage, and the term “progenitor ATZ cells” means progenitor cells derived from the ATZ crypt cells.

As used herein, the term “multipotent cells” refers to stem cells with the potential to differentiate into at least two cell types or lineages, and the term “multipotent ATZ cells” means multipotent cells derived from the ATZ crypt cells.

As used herein, the term “pluripotent cells” refers to stem cells with the potential to differentiate into each of the three primary groups of cells, i.e., ectoderm, mesoderm and endoderm, and the term “pluripotent ATZ cells” means multipotent cells derived from the ATZ crypt cells.

As used herein, the term “isolated ATZ stem cells” refers to a mixed population of stem cells (e.g., pluripotent stem cells, multipotent stem cells, and progenitor cells) derived from the ATZ crypts that have been isolated (e.g., physically, chemically, or enzymatically dissociated) to further isolate the ATZ crypt cells from the ATZ crypts. Alternatively, or in addition, the ATZ stem cells can be numerically expanded relative to other ATZ cells by one or more rounds of replating/passaging because of their superior properties of stem cell self-renewal.

As used herein, the terms “proliferation” and “proliferating” refer to an increase in cell number by mitosis.

As used herein, the term “differentiation” refers to the formation of cells expressing markers known to be associated with cells that are more specialised and closer to becoming terminally differentiated cells incapable of further division or differentiation.

For example, in a haematological context, differentiation can be seen in the production of functional cells of multiple cellular lineages (e.g., red blood cells, platelets, granulocytes, macrophages). The terms “further” or “greater” differentiation refers to cells that are more specialised and closer to becoming terminally differentiated cells incapable of further division or differentiation than the cells from which they were cultured.

As used herein, the term “terminally differentiated” refers to cells that are incapable of further differentiation and/or further division/proliferation or differentiation.

As used herein, the term “expanded” when referring to cells, means cells that have been increased in number by proliferation and differentiation in vitro.

As used herein, the term “minimal growth medium” or “MGM” refers to a cell culture medium which provides the nutrients necessary for ATZ-derived cell maintenance without promoting differentiation of multipotent ATZ-derived stem cells to an extent that results in a decreased absolute number of multipotent ATZ-derived stem cells.

As used herein, the term “lineage-specific medium” or “LSM” refers to a cell culture medium which provides the nutrients and growth factors necessary for multipotent ATZ-derived stems to undergo differentiation toward specific-lineage cells, and to result in a decreased absolute number of multipotent ATZ-derived stem cells. The lineage-specific differentiation may be partial (e.g., toward endoderm, mesoderm or ectoderm progenitors) or terminal (e.g., toward goblet cells, cardiac muscle, neurons).

As used herein, the term “eATZ stem cells” refers to an expanded population of ATZ stem cells that can be generated in suspension or adherent 2 D cultures (e.g., individual cells) (e.g., clusters) or 3D cultures consisting of complex structures (e.g., organoids).

The expanded population of cells may retain the ability to differentiate into one or more cell types. In some embodiments, the eATZ cells retain at least one marker of ATZ stem cells selected from the group consisting of CD34, CD117 (KIT), CD184 (CXCR4), OCT4, NANOG, SOX17, Brachyury (TBXT), PAX6 and NESTIN.

The expanded population of eATZ cells can be differentiated in LSM to generate a variety of organoids, including intestinal organoids or ATZ stem cell-derived pancreatic organoids or hepatic organoids or lung organoids for stem cell transplantation.

As used herein, the term “organoids” refers to a multiplicity of cells grown in culture which self-organize to form a three-dimensional structure. Organoid structures may be a simple solid mass of cells, a hollow mass (e.g., a cystic organoid), a tube, or a more complicated structure (e.g., a crypt-like or follicle-like structure).

As used herein, the term “cellular composition” refers to a preparation of cells, which preparation may include, in addition to the cells, non-cellular components such as cell culture media, e.g., proteins, amino acids, nucleic acids, nucleotides, co-enzyme, antioxidants, metals and the like. Furthermore, the cellular composition can have components which do not affect the growth or viability of the cellular component, but which are used to provide the cells in a particular format, e.g., as polymeric matrix for encapsulation or a pharmaceutical preparation.

As used herein, the term “phenotype” refers to the observable or detectable characteristics of a cell, such as size, morphology, RNA expression, protein expression, or other properties.

As used herein, the term “marker” refers to a biological molecule that can be used to identify the phenotype of a cell by its presence, absence, or concentration, or by its activity, inactivity, or level of activity.

As used herein, the term “exogenous biocompatible scaffolds” refers to three-dimensional porous, fibrous, or permeable volume-retaining biomaterials intended to provide physical or mechanical support while permitting diffusion or transport of body liquids and gases, allowing for cellular interactions. Preferably, such scaffolds cause limited or minimal inflammation and toxicity. Optionally, such scaffolds are biodegradable. Examples of scaffolds can include a biological scaffold (e.g., a laminin or collagen-based scaffold) and synthetic scaffolds (e.g., non-biological polymers such as PGA, PLA, PLGA). Scaffolds may be in the physical form of a thread, sheet, paste, powder, or liquid. Scaffolds can be used in vitro and in vivo.

As used herein, the phrase “pharmaceutically acceptable” refers to those compounds, materials, compositions, and/or dosage forms which are, within the scope of sound medical judgment, suitable for use in contact with the tissues of human beings and animals without excessive or unacceptable toxicity, irritation, allergic response, inflammatory response or other problem or complication, commensurate with a reasonable benefit/risk ratio.

As used herein, the phrase “pharmaceutically-acceptable carrier” means a pharmaceutically acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

As used herein, the term “Type 1 diabetes” refers to an autoimmune disease that originates when beta cells that make insulin are destroyed by the immune system. Insulin is a hormone required for cells to use blood glucose for energy and helps regulate glucose levels in the bloodstream. If left untreated, diabetes can cause many complications, including relatively rapid onset of diabetic ketoacidosis and nonketotic hyperosmolar coma. Long-term complications include heart disease, stroke, kidney failure, foot ulcers and damage to the eyes.

As used herein, the term “Type 2 diabetes” refers to a condition that is characterised by high blood sugar, relative lack of insulin and insulin resistance. Often symptoms develop slowly and may include increased hunger, feeling tired, and sores that do not heal. Long-term complications from high blood sugar include heart disease, strokes, diabetic retinopathy which can result in blindness, kidney failure, and poor blood flow in the limbs which may lead to amputations.

As used herein, the term “chronic liver disease” refers to several diseases of the liver including alcoholic liver disease, non-alcoholic steatohepatitis (NASH), or hepatitis C infection. These diseases may be associated with cirrhosis of the liver, a form of irreversible fibrotic scarring.

As used herein, the term “inflammatory bowel disease” is used to describe two conditions: ulcerative colitis and Crohn's disease, long-term conditions that involve inflammation of the gut. Ulcerative colitis only affects the colon (large intestine). Crohn's disease can affect any part of the digestive system, from the mouth to the anus.

As used herein, the term “lung diseases” refers to many disorders affecting the lungs, such as asthma, chronic obstructive pulmonary disease (COPD), infections (e.g., influenza, pneumonia, and tuberculosis), occupational lung diseases, and lung cancer.

As used herein, “significant levels” of a stem cell marker can be determined by standard quantitative methods which are suitable to the marker (e.g., quantitative detection of antibody or other ligand binding to cell surface proteins; quantitative detection of mRNA expression by quantitative PCR; quantitative detection of catalytic products for enzymes). For each method, a standard error of measurement or a standard level of background noise can be determined. If the measured level of expression is not statistically significant at the 5% level for the chosen methods, then the marker is not expressed at “significant levels.”

### Multipotent and Progenitor ATZ Cells and Organoid Compositions

The multipotent and/or progenitor ATZ cells described herein can be isolated as single cell (or multiple cell) preparations derived from primary ATZ tissue, including isolated ATZ crypt cells. The ATZ cells described herein, including multipotent and progenitor ATZ cells, can be isolated from ATZ crypts obtained by biopsy, or can be derived from organoid structures differentiated ex vivo from isolated ATZ crypt cells. In some embodiments, the multipotent and progenitor ATZ cells are obtained from organoids derived from single primary ATZ crypt cells or dissociated ATZ crypt cells.

For use in the methods of treatment described herein, the multipotent or progenitor ATZ cells are preferably autologous cells derived from a human patient or allogeneic cells derived from a human donor (preferably tissue-matched to reduce or avoid graft-versus-host or host-versus-graft immunoreactivity). Alternatively, the multipotent or progenitor ATZ cells can be derived from other mammalian species and can be modified by methods known in the art to reduce or eliminate alloreactivity (e.g., gene editing to knock-out MHC Class I and/or Class II genes and/or the 32-microglobulin gene).

In some embodiments, the multipotent or progenitor ATZ cells can be in vitro expanded ATZ (eATZ) cells.

In some embodiments, the multipotent and progenitor ATZ cells, primary ATZ cells, isolated ATZ crypt cells and/or ATZ-derived organoids are combined in vivo or in vitro with an exogenous biocompatible scaffold, for example, a synthetic scaffold or a biological scaffold. In some embodiments, the scaffold comprises laminin and/or collagen (e.g., Permacol™ Paste, Medtronic PLC, Minneapolis, MN). In some embodiments, the scaffold is Corning® Matrigel® Matrix (Corning, Inc, New York; Geltrex™ Growth Factor Basement Membrane Matrix, Thermo Fisher Scientific) or a functional equivalent.

In some embodiments, the scaffold comprises a functionalised collagen scaffold or a functional equivalent.

The exogenous biocompatible scaffold can also comprise cell culture medium components. In some embodiments, the scaffold comprises an endodermal lineage-specific medium (LSM), which can be purchased from commercial vendors (e.g., IntestiCult™, Human Intestinal Protocols, Bio-Techne); pancreas (e.g. PancreaCult™); liver (HepatiCult™); lung (e.g., STEMdiff™ Branching Lung Organoid Kit, or PneumaCult™ Airway Organoid Kit, or PneumaCult™ Apical-Out Airway Organoid Medium, or PneumaCult™ Alveolar Organoid Medium, all from STEMCELL Technologies, Inc; Human Lung Organoid Culture Protocols, Bio-Techne; 3dGRO™ Lung Organoid Branching Medium3dGRO™ and Lung Organoid Maturation Medium, Merck).

In some embodiments, the ATZ cells or ATZ crypt cells can be cultured in an MGM. In some embodiments, the ATZ cells or ATZ crypt cells can be cultured in a feeder-free medium that maintains human embryonic stem cells (ESCs) and/or induced pluripotent stem cells (iPSCs) in an undifferentiated state (e.g., mTesR™, STEMCELL Technologies). In some embodiments, the scaffold comprises such an ESC or iPSC feeder-free medium. In some embodiments, the ATZ cells or ATZ crypt cells cultured in LSM have a higher expression of mesoderm and endoderm lineage markers as compared to ATZ cells or ATZ crypt cells cultured in such an ESC or iPSC feeder-free medium. The matrix can also comprise keratinocyte serum-free medium (KSFM) for differentiation. Such KSFM medium is commercially available from vendors (e.g., Gibco™ Keratinocyte-SFM Medium, Thermo Fisher Scientific; DermaCult™ Keratinocyte Expansion Medium, STEMCELL Technologies).

In another aspect, the disclosure provides for phenotypic characterisation of extracellular and intracellular protein and mRNA expression levels of stem cell and progenitor cell markers on freshly isolated ATZ cells. ATZ cells can express at least one of CD34, CD117, and CD184 cell surface markers but do not express detectable levels of CD45 as detected by flow cytometry. In some embodiments, isolated porcine ATZ cells express OCT4 and NANOG.

ATZ stem cells can also express markers for stem cells of three developmental lineages: endoderm (SOX17), mesoderm (TBXT) and ectoderm (PAX6 and NESTIN). In addition, freshly isolated ATZ cells can express markers for pluripotent stem cells (NANOG and OCT4).

In some embodiments, the ATZ cells and/or crypts express genes associated with endoderm, ectoderm, and mesoderm lineages.

In some embodiments, the ATZ cells and/or crypts express CD34. In some embodiments, the ATZ cells and/or crypts do not express detectable levels of CD45. In some embodiments, the ATZ cells and/or crypts express CD34 and do not express detectable levels of CD45.

In some embodiments, the ATZ cells and/or crypts express at least one marker selected from the group consisting of ALP, TBXT, BMP4, CD34, KIT, CXCR4, CHGA, CK18, EPCAM, LGR5, LYS, MUC2, NANOG, PAX6, SOX17, and OCT4. In some embodiments, the ATZ cells and/or crypts express at least 2, 3, 4, 5, 6, 7, 8, 9, 10, 11, 12, 13, 14, 15 or 16 markers selected from the group consisting of ALP, TBXT, BMP4, CD34, KIT, CXCR4, CHGA, CK18, EPCAM, LGR5, LYS, MUC2, NANOG, PAX6, SOX17, and OCT4.

In some embodiments, the ATZ cells and/or crypts express at least one marker selected from the group consisting of ALP, TBXT, BMP4, CD34, KIT, CXCR4, CHGA, CK18, EPCAM, LGR5, LYS, MUC2, PAX6, and SOX17. In some embodiments, the ATZ cells and/or crypts express at least 2, 3, 4, 5, 6, 7, 8, 9, 10, 11, 12, 13 or 14 markers selected from the group consisting of ALP, TBXT, BMP4, CD34, KIT, CXCR4, CHGA, CK18, EPCAM, LGR5, LYS, MUC2, PAX6 and SOX17. In some embodiments, the ATZ cells and/or crypts express OCT4 or NANOG genes.

In some embodiments, the ATZ cells and/or crypts express CD117 (KIT) at a higher level as compared to small intestine, colon, and rectum crypt cells. In some embodiments, the ATZ cells and/or crypts express CD117 (KIT) at least 1.5-fold, 2-fold, 2.5-fold, 3-fold, 3.5-fold, 4-fold, 4.5-fold, 5-fold, 5.5-fold, 6-fold, 6.5-fold, 7-fold or higher as compared to small intestine, colon, and rectum crypt cells.

An aspect of the disclosure provides for phenotypic characterisation of extracellular and intracellular protein and mRNA expression levels of stem cell and progenitor cell markers on ATZ organoids in culture. In some embodiments, the ATZ organoids express genes associated with endoderm, ectoderm, and mesoderm lineages. In some embodiments, the ATZ organoids express at least one protein marker selected from the group consisting of alkaline phosphatase, brachyury, CD34, CD117 (KIT), chromogranin, cytokeratin 18, CD184 (CXCR4), EpCAM, GD2, LGR5, lysozyme, mucin 2, Nestin, and PAX6. In some embodiments, the ATZ organoids express CD34. In some embodiments, the ATZ organoids do not express detectable levels of CD45. In some embodiments, the ATZ organoids express at least one gene selected from the group consisting of ALP, TBXT, BMP4, CD34, KIT, CXCR4, CHGA, CK18, EPCAM, LGR5, LYS, MUC2, NANOG, PAX6, and SOX17.

In some embodiments, the ATZ organoids express at least one cell surface marker selected from the group consisting of CD31, CD34, CD117 (KIT), CXCR4 (CD184), CK14, CK15, CK18 GD2, SOX2, SSEA4, and TRA-1-60. In some embodiments, the ATZ organoids express CD34. In some embodiments, the ATZ organoids do not express detectable levels of CD45. In some embodiments, the ATZ organoids express CD34 but do not express detectable levels of CD45.

In some embodiments, the ATZ organoids express CD117 (KIT), CXCR4 (CD184), and/or GD2 at a higher level as compared to small intestine and rectum organoids. In some embodiments, the ATZ organoids express CD117 (KIT) at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to small intestine and rectum organoids. In some embodiments, the ATZ organoids express CXCR4 (CD184) at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to small intestine and rectum organoids. In some embodiments, the ATZ organoids express GD2 at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to small intestine and rectum organoids. In some embodiments, the ATZ organoids have reduced SOX17 expression as compared to ATZ crypt cells.

In some embodiments, the ATZ organoids have reduced TBXT expression as compared to ATZ crypt cells. In some embodiments, the ATZ organoids have reduced CD31 expression as compared to ATZ crypt cells. In some embodiments, the ATZ organoids have reduced PAX6 expression as compared to ATZ crypt cells. In some embodiments, the ATZ organoids have increased LGR5 as compared to ATZ crypt cells. In some embodiments, the ATZ organoids have increased CD117 or CXCR4 as compared to ATZ crypt cells. In some embodiments, the ATZ organoids have increased K14 as compared to ATZ crypt cells. In some embodiments, the ATZ organoids have increased expression of CD117, CXCR4, LGR5, or K14 of at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to ATZ crypt cells. In some embodiments, the ATZ organoids have decreased expression of CD31, PAX6, SOX17, or TBXT 1.5-fold, 2-fold, 2.5-fold, 3-fold, 3.5-fold, 4-fold, 4.5-fold, 5-fold, 5.5-fold, 6-fold, 6.5-fold, or 7-fold lower as compared to ATZ crypt cells.

Protein and nucleic acid expression can be determined by methods known to one of skill in the art. Such methods include, but are not limited to, polymerase chain reaction (PCR), RTPCR, q-RT-PCR, flow cytometry (combined with binding agents (e.g., labelled antibodies or ligands for cell surface markers), SDS-PAGE, mass spectrometry, immunoblotting (Western blotting), immunofluorescence microscopy, fluorescence in situ hybridisation, or any other technique known in the art.

In some embodiments, the ATZ organoids comprise proliferating cells, secretory cells, and cytokeratin. In some embodiments, the ATZ organoids can secrete lysozyme.

### Methods of Isolating and Producing Populations of Multipotent and Progenitor ATZ Cells

In another aspect, provided herein are methods of isolating and producing multipotent and/or progenitor ATZ cells. The ATZ cells can be produced by obtaining anorectal transition zone tissue from a mammalian subject (e.g., human, porcine) either from biopsied or resected tissue; washing tissues with a serum-free tissue culture medium optionally containing antibiotics and/or antimycotics; mincing the tissue; and enzymatically treating or digesting the tissues with a collagenase. Digestion can be stopped by, for example, adding protein such as albumin. Crypts can be isolated by rigorously stirring or shaking the tissue digest to release the crypts. Medium containing crypts can be passed through tissue strainers to remove large debris and centrifuged to sediment or pellet crypts; and the crypts can be resuspended in fresh medium to obtain a population of multipotent and/or progenitor ATZ cells.

Resuspended crypts can be enumerated under a microscope. Single cell preparations from crypts are generated by physical disruption with a needle/syringe and/or treatment with mild enzymatic cell dissociation reagents to obtain a population of multipotent and/or progenitor ATZ cells. These cells can then be embedded in an in vitro matrix scaffold (for example, as described above), and cultured with differentiation media to produce organoids.

In some embodiments, the enzymatic treatment of minced ATZ tissue releases intestinal crypts and submucosal glands. In some embodiments, the anorectal transition zone tissue is from a healthy human donor. In some embodiments, the anorectal transition zone tissue is from a diseased human donor.

The released intestinal crypts or multipotent and/or progenitor ATZ cells can then be differentiated into organoids after being placed in a growth medium or a scaffold (e.g., laminin, collagen, Permacol™, Corning® Matrigel® Matrix). Any suitable matrix and growth medium know in the art can be used. In some embodiments, the crypts are placed in a synthetic scaffold. In some embodiments, the scaffold is non-functionalised. In some embodiments, the scaffold is functionalised. Alternatively, the crypts can be further dissociated into single ATZ cells.

In some embodiments, the isolated ATZ-derived crypt cells have increased multipotent and/or progenitor capacity as compared to cells derived from rectum crypts.

In another aspect, the invention provides for in vitro maintenance of ATZ stem cells (including multipotent and/or progenitor cells) by culturing crypts or single cells embedded in a scaffold (e.g., Permacol™, Corning® Matrigel® Matrix) with a minimal growth medium (MGM). The use of MGM permits maintenance of the percentage of multipotent ATZ stem cells (+/−10%) without promoting terminal differentiation the cells. To be clear, in a mixed culture of ATZ cells, including ATZ-derived crypt cells and/or ATZ-derived organoid cells, there will be multipotent ATZ stem cells, ATZ progenitor cells, and partially or terminally differentiated ATZ cells, and some cells will become more differentiated over time. However, if the rate of multipotent ATZ stem cell proliferation equals or exceeds the rate of differentiation (and loss of multipotency), the ATZ stem cell culture can be maintained indefinitely. If the rate of multipotent ATZ stem cell proliferation is slightly exceeded by the rate of differentiation (and loss of multipotency), the ATZ stem cell culture can nonetheless be maintained for several weeks, which is sufficient for the methods of treatment described herein.

Preferably, ATZ organoid cultures are refreshed on periodic basis by breaking-up organoids and replating the ATZ-derived organoid cells in MGM. Using an appropriate MGM, it is possible to maintain at least 5%, at least 10% or at least 15% multipotent ATZ stem cells after at least 3 weeks, 4 weeks, at least 5 weeks, or at least 6 weeks of organoid culture. For example, the methods described herein allow at least 5% multipotent stem cells to be obtained after 5 passages, 10% after 10 passages, and 10% after 20 passages. Percentages of multipotent ATZ stem cells are relative to total ATZ cells.

In another aspect, the invention provides for methods of in vitro expansion of ATZ stem cells (including multipotent and/or progenitor cells) in order to produce sufficiently large populations of cells (e.g., 10³, 10⁴, 10⁵, 10⁶, 10⁷, 10⁸) to be useful for cell transplantation into damaged or dysfunctional endodermal tissues. The methods can include repeated passaging of ATZ organoids in MGM. To be clear, during expansion, the absolute number of multipotent ATZ-derived stem cells can increase while the percentage of multipotent ATZ-derived stem cells decreases. For purposes of transplantation into a damaged or dysfunctional, the absolute number of cells will be more relevant in many applications. The methods of expansion can further include confirmation that harvested cells maintain desired phenotypic properties determined, for example, by protein and mRNA expression levels of cell surface and intracellular stem cell markers and replating efficiencies. Once the properties of ATZ cells are validated for a cell source, a biomarker such as a cell surface marker (e.g., CD34, CD117, and/or CD184) can be used to determine appropriate numbers of cells for stem cell therapy.

Examples of commercially available MGM include mTesR™ (STEMCELL Technologies, Inc, Vancouver, CA) and StemFit® Feeder-Free Stem Cell Culture Media (AMSBIO, Abingdon, UK). In addition, despite its name, IntestiCult™; Human Intestinal Organoid Culture Protocol (Bio-Techne, Minneapolis, MN) can be used as an MGM at lower concentrations because the rate of multipotent ATZ stem cell proliferation can equal or exceed the rate of differentiation (and loss of multipotency).

In some embodiments, a mixed culture of ATZ-derived stem cells and ATZ-derived organoids, including multipotent ATZ-derived stem cells, can be induced to differentiate toward endodermal tissue by culturing the cells in an endodermal lineage-specific medium (LSM). The resulting mixed population of cells will include a greater percentage of ATZ-derived endodermal progenitor cells which can be used in the methods of treatment described herein.

Examples of commercially available endoderm LSMs include PancreaCult™ (pancreas); HepatiCult™ (liver); STEMdiff™ Branching Lung Organoid Kit, or PneumaCult™ Airway Organoid Kit, or PneumaCult™ Apical-Out Airway Organoid Medium, or PneumaCult™ Alveolar Organoid Medium, Human Lung Organoid Culture Protocols, Bio-Techne; 3dGRO™ Lung Organoid Branching Medium3dGRO™ and Lung Organoid Maturation Medium, Merck (lung). In addition, IntestiCult™; Human Intestinal Organoid Culture Protocol (intestine) can be used as an LSM at higher concentrations because the rate of differentiation (and loss of multipotency) can exceed the rate of multipotent ATZ stem cell proliferation.

An aspect of the disclosure provides for cryopreservation and thawing of ATZ cells after harvesting ATZ crypts, single cells and/or after organoid culture by resuspending centrifuged cells in serum-free, DMSO-containing freezing medium and gradually decreasing the temperature to −80° C. or lower. To re-establish organoid cultures, cryopreserved ATZ organoids or single cells are quickly thawed in a 37° C. water bath, resuspended in pre-warmed tissue culture medium, and centrifuged and resuspended in fresh medium to remove freezing medium. In some embodiments, the composition further comprises cryopreservation media. In some embodiments, the ATZ or eATZ composition is cryopreserved.

In some embodiments, ATZ-derived endodermal-derived organoids (e.g., intestinal, pancreatic, liver, lung) are generated from biopsies taken from a patient with an abnormal functioning tissue or organ.

### Methods of Treatment

In some embodiments, the ATZ crypts are cultured ex vivo to produce eATZ cells for use in a method of treating tissues and organs of endodermal origin. In some embodiments, the eATZ cells can be cryopreserved after expansion and then thawed prior to treatment.

In some embodiments, the pharmaceutically acceptable carrier comprises an exogenous biocompatible scaffold, for example, a scaffold described hereinabove.

In another aspect, provided herein are methods of preparing a therapeutic or pharmaceutical composition (e.g., a cellular composition as disclosed herein). The method comprises (a) harvesting ATZ tissue from a patient or donor; (b) enzymatically digesting the ATZ tissue with an enzyme to prepare a cell suspension; (c) optionally combining at least a portion of the cell suspension with a cryopreservation media and cryopreserving the cell suspension; and (d) combining the cell suspension of step (b) or optional step (c) with a pharmaceutically acceptable carrier.

In one embodiment, the disclosure provides a cellular composition comprising adult allogeneic or autologous ATZ cells in a pharmaceutically acceptable carrier. The ATZ cells can be prepared by a method that comprises: (a) collecting ATZ tissue from an adult human or porcine subject; (b) preparing a cell suspension in vitro by enzymatic digestion of ATZ tissue; (c) sedimenting/pelleting and then re-suspending the cells in freezing medium and cryopreserving ATZ cells in, for example, liquid nitrogen. Prior to use, the cryopreserved ATZ cells are combined with a pharmaceutically acceptable carrier, and the cell/carrier preparation is then used to treat a damaged or dysfunctional endodermal tissue (e.g., pancreas, liver). The carrier can be used to provide support for ATZ cells in an endodermal tissue or organ.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present disclosure remain operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

### EXAMPLES

The following examples are merely illustrative and are not intended to limit the scope or content of the disclosure in any way.

### Example 1. Isolation of Intestinal Crypts from Anorectal Tissue

This example describes protocols for isolating intestinal crypts from anorectal tissue from a pig and a human subject with Crohn's disease.

### I. Pig

An example of healthy adult white Landrace porcine tissue from the colon to anus (FIG. 1(a)) was collected in AIMV V medium (Thermo Fisher Scientific, Waltham, Massachusetts) containing antibiotics/antimycotics within 2 hours of termination and processed directly, or in some instances after overnight storage in at 4° C. The tissue was longitudinally opened and cleaned.

The dentate line was identified between the rectal mucosa and anal skin (FIG. 1(b)). The width of the tissue was 8-10 cm with a height of 1-2 cm and was pale in colouration. The epithelial layer of dentate tissue (anorectal transition zone) was excised from rectal mucosa and anal skin (FIG. 1(c)). Enzymatic treatment of minced anorectal transition zone tissue released intestinal crypts (FIG. 1(d-g)) and structures consistent with submucosal glands (FIG. 1(h-i)).

### II. Crohn's Disease Patient

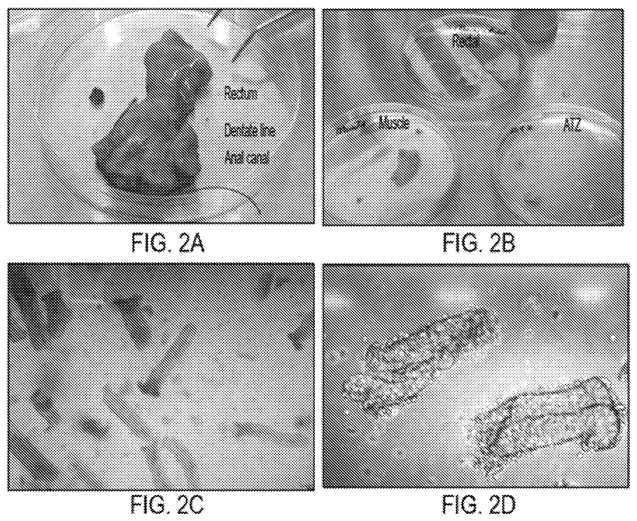

From a proctectomy tissue (FIG. 2(a)), the anorectal transition zone was excised (FIG. 2(b)), minced and enzymatically treated to release crypts (FIG. 2(c-d)).

### Example 2. Crypt Anorectal Transition Zone Organoid Development

Crypt anorectal transition zone organoid development from the pig and human tissue of EXAMPLE 1 was developed.

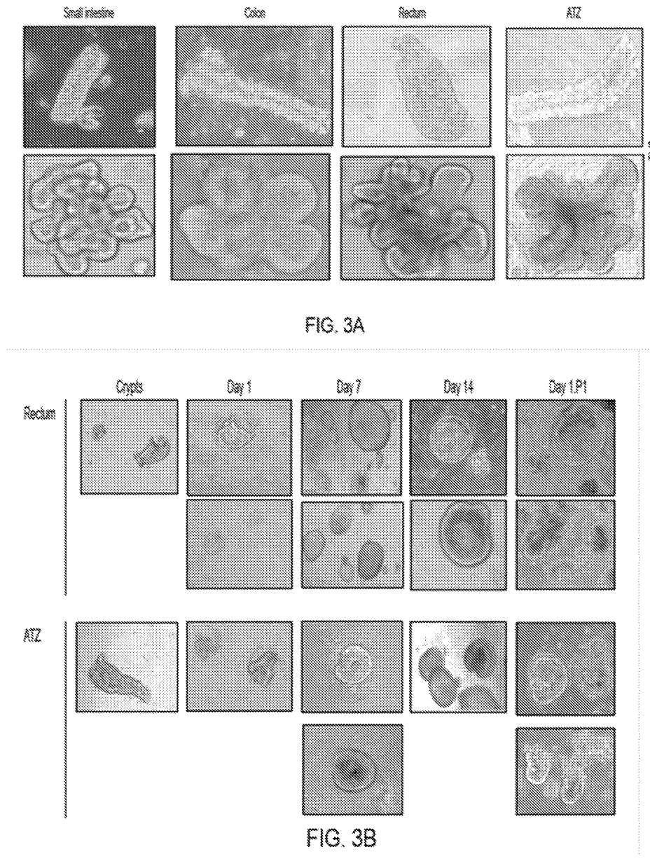

Intestinal crypts prepared in EXAMPLE 1 were embedded in Corning® Matrigel® Matrix with human IntestiCult™ and developed into fully branched organoids within 1-2 weeks. The morphology of porcine crypt organoids derived from anorectal transition zone were indistinguishable from organoids derived from porcine small intestine, colon, and rectum tissue (FIG. 3(a)). Similarly, organoids derived from anorectal transition zone and rectum were indistinguishable in a Crohn's disease patient (FIG. 3(b)).

### Example 3. Increased Progenitor Capacity of Single Cell Preparations of Porcine ATZ Crypt Cells Compared to Rectal Crypt Cells

This example demonstrates that single cell preparations of porcine ATZ crypt cells have increased progenitor capacity, assessed by plating efficiency, compared to rectal crypt cells for the purpose of describing and illustrating certain examples and embodiments of the present disclosure.

The plating efficiencies (organoids formed per cells plated) of single cell preparations of porcine ATZ and rectal crypt cells were determined for an input of 500, 1,000 and 2,000 viable crypt cells per well in 24 well, flat base, suspension surface culture plates (Sarstedt, Nümbrecht, Germany) and monitored weekly for 4 weeks. IntestiCult™ was refreshed once at the end of week 2 without a change in Corning® Matrigel® Matrix.

Few organoids formed from 500 input cells in either group.

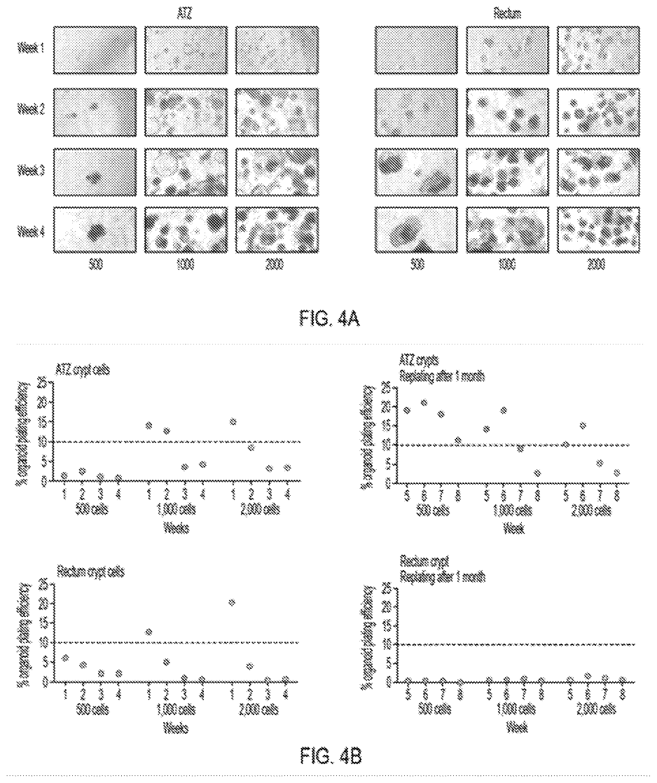

At week 1, the appearance (FIG. 4(a)) and frequency (FIG. 4(b)) of organoids forming was similar for ATZ-derived and rectal-derived organoids for cultures initiated with 1,000 or 2,000 cells. At week 2, rectal-derived organoid cultures contained more fully differentiated organoids (dark clusters of cells), whereas ATZ-derived organoid cultures contained more undifferentiated viable organoids (cystic organoid structures) (FIG. 4(a)).

Few viable organoids were detected at weeks 3 and 4 in cultures initiated with 1,000 and 2,000 cells: 3-4% for ATZ and 0-1% for rectum (FIG. 4(b)).

After 4 weeks, cultures were harvested and reinitiated with 500, 1,000, or 2,000 viable cells to determine plating efficiencies for an additional 4 weeks. IntestiCult™ was refreshed at week 6 without a change in Corning® Matrigel® Matrix. Plating efficiencies were assessed at weeks 5, 6, and 7 (FIG. 4(b)). The highest plating efficiency was observed with 500 replated ATZ-derived cells over weeks 5-7 (18%-21%). Plating efficiencies for 1,000 and 2,000 replated ATZ-derived cells peaked at week 6 (19% and 15%, respectively) and decreased at week 7 (9% and 5%, respectively).

Plating efficiencies for all replated rectal-derived cells was <2% over weeks 5-7. Taken together, these studies show increased progenitor capacity, determined by plating efficiencies, in porcine ATZ crypt cells compared to rectum crypt cells. These results are consistent with the existence of a long-term repopulating ATZ stem cell population.

### Example 4. Protein Expression of Stem Cell Markers of Embryonic Developmental Lineages on Freshly Isolated ATZ Crypts and ATZ-Derived Crypt Organoids

This example shows protein expression detected by immunocytochemistry of stem cell markers of embryonic developmental lineages on freshly isolated porcine crypts and crypt organoids for the purpose of describing and illustrating certain examples and embodiments of the present disclosure.

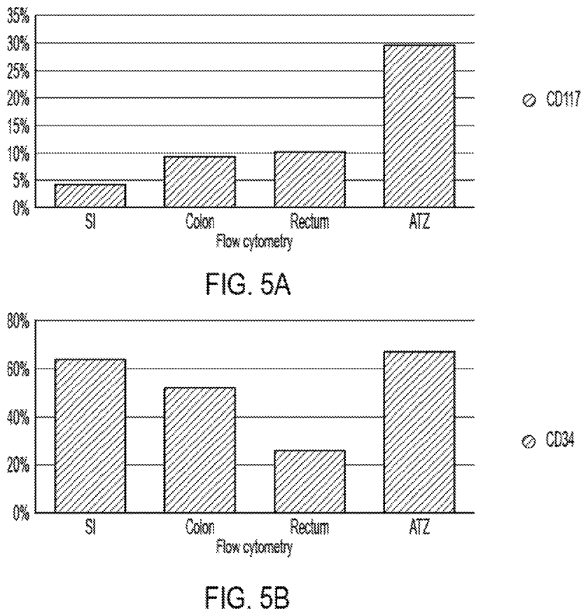

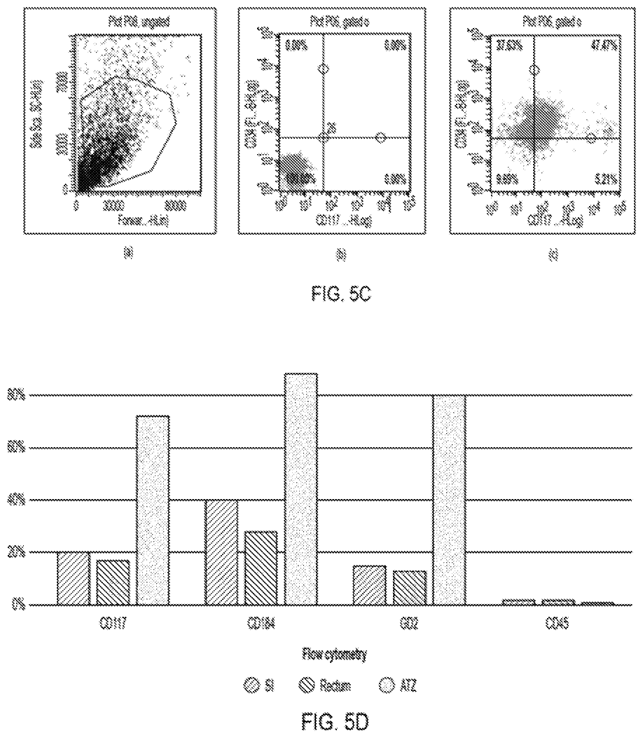

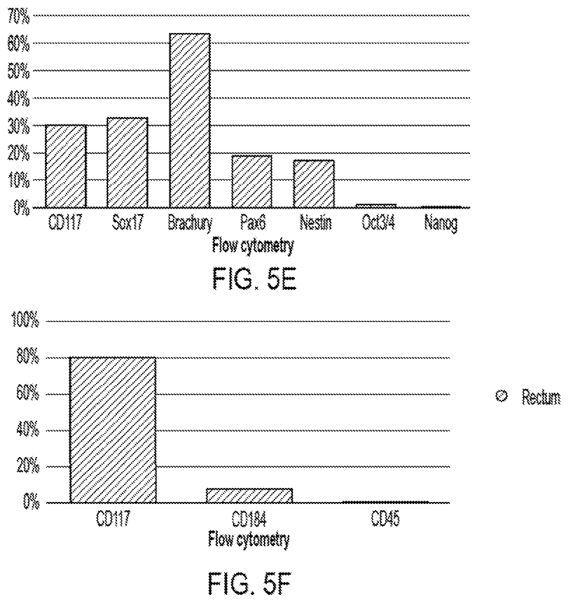

Freshly isolated porcine ATZ crypts. Cell surface stem cell markers were examined by flow cytometry on single cell preparations from freshly isolated porcine crypts derived from the small intestine, colon, rectum, and ATZ tissue. Expression of the stem cell and progenitor cell growth factor receptor, KIT (CD117), was expressed at 3-5-fold higher levels in ATZ crypt cells compared to small intestine, colon, and rectum crypt cells (FIG. 5(a)). Crypt cells from these tissues all expressed the stem cell and progenitor cell marker, CD34, to varying extent, with ATZ crypt cells expressing the highest levels (FIG. 5(b)).

In a double labelling experiment, 85% of ATZ crypt cells expressed CD34, 53% of cells expressed CD117; 47% of cells co-expressed CD34 and CD117; and of the CD117 expressing cells, 88% co-expressed CD34 (FIG. 5(c)).

Porcine ATZ crypt organoids. Cell surface stem cell markers were examined by flow cytometry on single cell preparations of fully differentiated porcine crypt organoids derived from the small intestine, rectum, ATZ cells after 10 days of culture. Expression of CD117, CD184 and GD2 were increased by more than 3-fold in ATZ-derived organoids compared to SI- and rectal-derived organoids. The haematopoietic marker, CD45, was undetected in all crypt organoids (FIG. 5(d)).

Because KIT and CXCR4 are markers of definitive endoderm and GD2 is a marker of primitive mesoderm, further investigation of the developmental lineage origins of freshly isolated ATZ crypt cells was undertaken. Intracellular protein markers confirmed the expression of transcriptional factors associated with the endoderm (SOX17), mesoderm (TBXT), and ectoderm (PAX6 and NESTIN) lineages. However, expression of transcriptional factors associated with pluripotency (OCT4 and NANOG) were not detected by flow cytometry (FIG. 5(e)).

Taken, together these results indicate porcine ATZ crypt cell populations express stem cell and progenitor protein markers for all 3 developmental lineages, and that markers for endoderm and mesoderm were maintained in ATZ crypt organoids.

### Example 5. MRNA Expression Profiling of Porcine ATZ Crypt Cells

This example shows mRNA expression profiling of porcine ATZ crypt cells for the purpose of describing and illustrating certain examples and embodiments of the present disclosure.

### Fresh Porcine ATZ Crypt Cells.

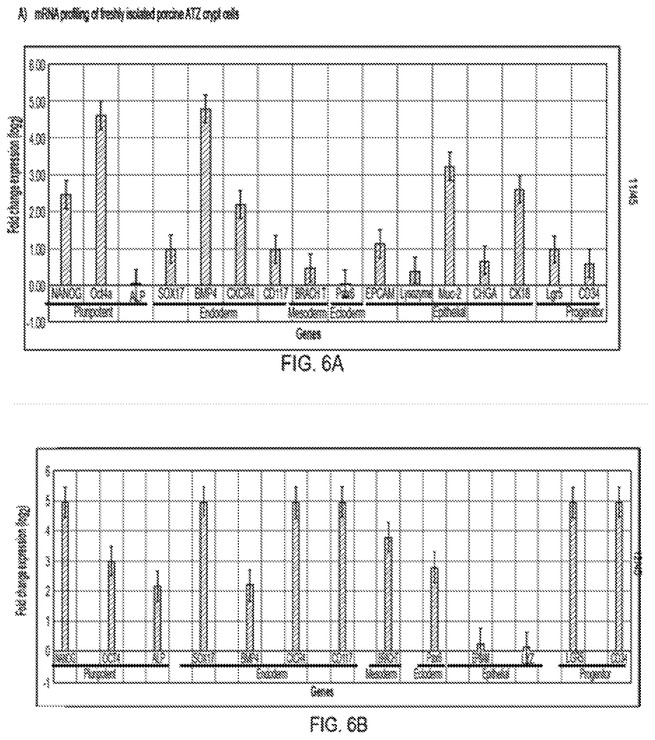

Transcriptional profiling of fresh porcine ATZ crypt cells confirmed basal expression of markers for mature epithelial cells (EPCAM, LYZ, MUC2, CHGA, CK18); stem cells and progenitor cells (CD34, LGR5); and developmental stem cells of the endoderm (SOX17), mesoderm (TBXT), and ectoderm (PAX6) lineages; and pluripotent stem cells (NANOG, OCT4A) (FIG. 6(a)). Although mRNA expression of alkaline phosphatase (ALP) was not detected, the enzyme was detected by immunocytochemistry in

### Porcine ATZ-Derived Crypt Organoids.

Transcriptional profiling of ATZ-derived crypt cells cultured in IntestiCult™ retained mRNA expression for markers of stem cells of all three developmental lineages (FIG. 6(b)). Lower levels of mRNA expression for markers of mature epithelial cells were detected when compared to fresh ATZ cells. mRNA expression levels were normalised to glyceraldehyde 3-phosphate dehydrogenase (GAPDH).

Taken together, transcriptional profiling of fresh ATZ crypts and organoids confirmed the presence of markers for stem cells of all three developmental lineages.

### Example 6. Culture Medium for Maintaining and Expanding Multipotent Porcine ATZ Stem Cells

This example demonstrates in vitro culture conditions for maintaining and expanding multipotent ATZ cells for the purpose of describing and illustrating certain examples and embodiments of the present disclosure.

Freshly isolated porcine ATZ crypt single cells were cultured for 14 days in either (1) a feeder-free medium that maintains human embryonic stem cells (ESCs) and induced pluripotent stem cells (iPSCs) in an undifferentiated state (mTeSR™) or in (2) human intestinal IntestiCult™.

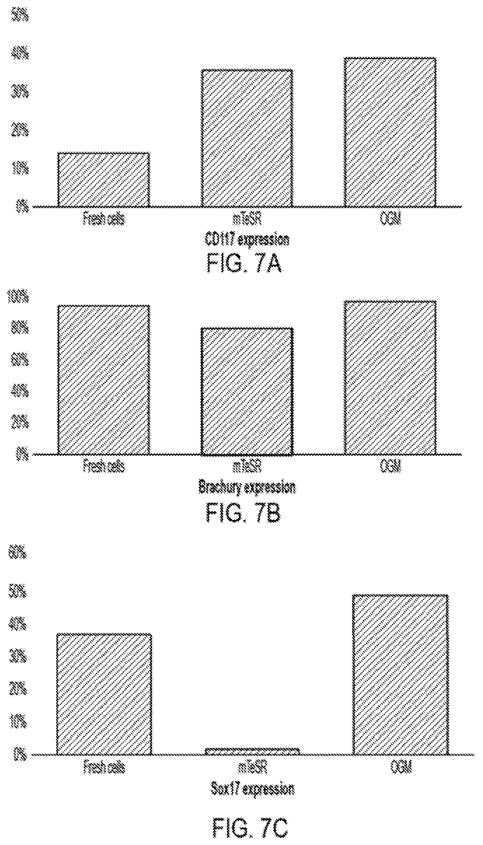

The protein expression levels of multipotential stem cell markers described in EXAMPLE 4 were assessed by flow cytometry before culture (day 0) and after 14 days in culture. The stem cell and progenitor marker CD117 was expressed on 14% ATZ crypt cells before culture (day 0) and increased to 35% in mTeSR™ and 28% human IntestiCult™ at day 14 (FIG. 7(a)).

The intracellular marker for mesoderm (Brachyury) was expressed on 97% of ATZ crypt cells before culture (day 0) and the expression levels were maintained at 99% in mTeSR™ and 81% in human IntestiCult™ at day 14 (FIG. 7(b)).

The intracellular marker for endoderm, SOX17, was expressed on 34% of ATZ crypt cells before culture (day 0) and <1% expression in mTeSR™ medium and 49% in human IntestiCult™ at day 14 (FIG. 7(c)).

These results indicate that IntestiCult™ may be preferable for maintaining ATZ crypt cells expressing markers of both endoderm and mesoderm developmental lineages.

### Example 7. In Vitro Differentiation of Porcine ATZ Cell-Derived Crypts into all Three Developmental Lineages

This example demonstrates that porcine ATZ-derived crypts differentiate in vitro into all three developmental lineages (endoderm, mesoderm, and ectoderm) for the purpose of describing and illustrating certain examples and embodiments of the present disclosure.

Expression of protein and mRNA markers in all three developmental lineages of fresh porcine ATZ crypt stem cells in EXAMPLE 4 and EXAMPLE 5, respectively, led to studies to determine if freshly isolated ATZ crypt cells had the capacity to generate mature cells of the endoderm lineage (gut mucosa), mesoderm (blood vessels), and ectoderm (keratinocytes).

### Example 7.1. Endoderm Differentiation Potential of Fresh Porcine ATZ Crypts

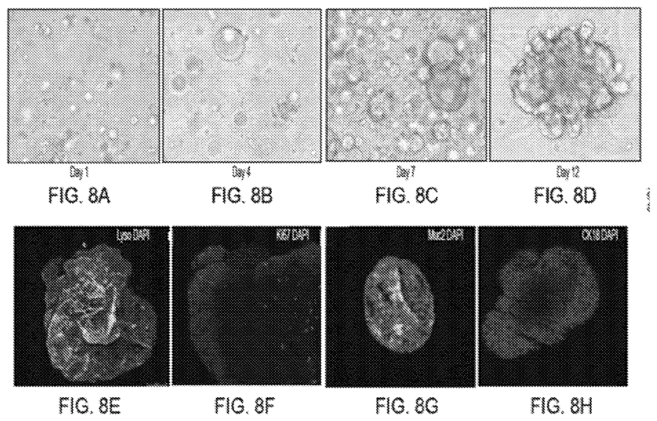

Protein expression levels of mature endoderm cell markers of gut mucosa were assessed after two weeks of culturing single cell preparations of fresh ATZ crypts embedded in Corning® Matrigel® Matrix with human IntestiCult™ to generate organoids. Immunostaining of intact organoids was visualised for expression of lysozyme, proliferating cells (KI67), secretory cells (MUC2), and cytokeratin 18 (CK18) which is expressed in the single layer of gut epithelium (FIG. 8(a-h)).

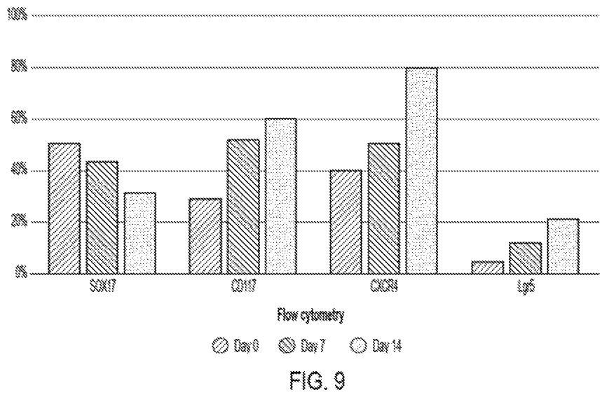

Flow cytometric analysis of fresh ATZ crypt cells showed that SOX17 decreased from 52% at day 0 to 34% at day 14; and that KIT (CD117) and CXCR4 (CD184) increased by twofold during culture (FIG. 9). The intestinal stem cell marker, LGR5, was expressed at 3% on day 0 and increased to 22% at day 14.

Taken together these results support that fresh ATZ crypts have the capacity to generate mature cell types derived from the endodermal lineage.

### Example 7.2. Mesoderm Differentiation Potential of Porcine ATZ Crypts

Protein expression levels of mature mesoderm cell markers were assessed after two weeks of culturing single cell preparations of fresh ATZ crypts in MethoCult™ to test their capacity to generate progeny in the haematopoietic lineage. No haematopoietic colonies formed in the non-adherent, methylcellulose layer.

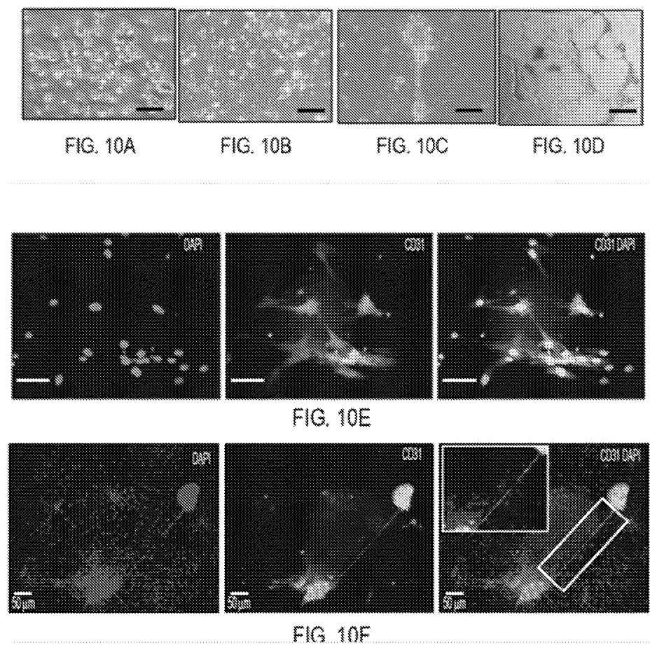

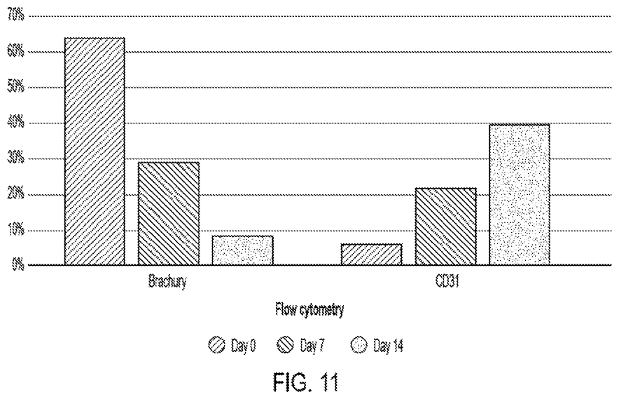

However, single ATZ crypt-derived cells (FIG. 10(a)) formed clusters in the adherent layer at day 3 (FIG. 10(b)) and initiated and formed a network of blood vessel-like tubular structures at day 10 (FIG. 10(c-d)). Immunostaining with the endothelial intracellular marker, CD31 (PECAM), was detected at the initiation and formation of a network of blood vessel-like tubular structures (FIG. 10(e-f)). Flow cytometric analysis of fresh ATZ cells showed that brachyury expression decreased from 67% at day 0 to 23% at day 7 and decreasing further to 7% on day 14; CD31 expression increased from 5% at day 0 to 19%, and further at day 7 to 37% at day 14 (FIG. 11).

Taken together these results support that fresh ATZ crypts have the capacity to generate mature cell types derived from the mesoderm lineage.

### Example 7.3. Ectoderm Differentiation Potential of Porcine ATZ Crypts

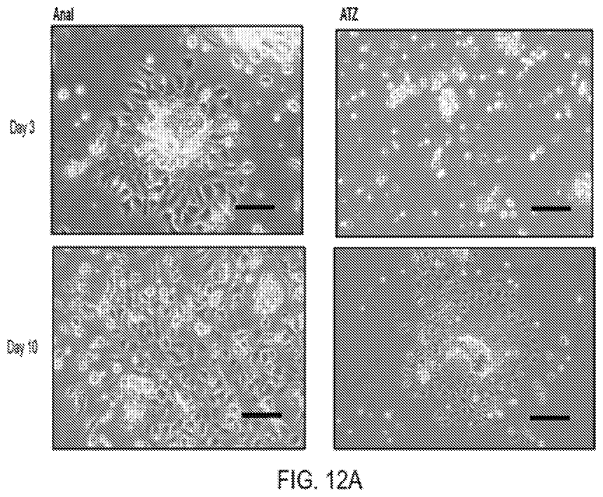

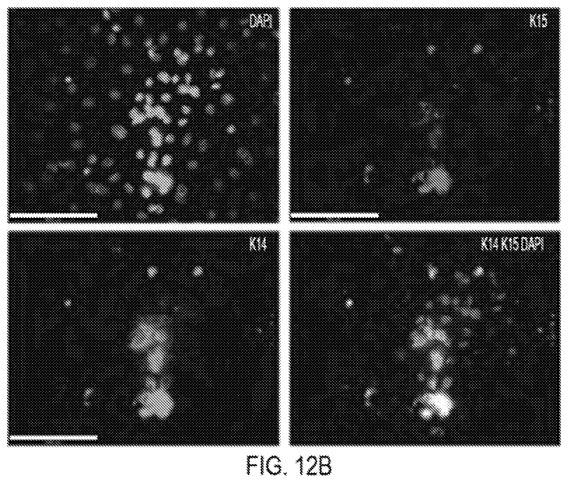

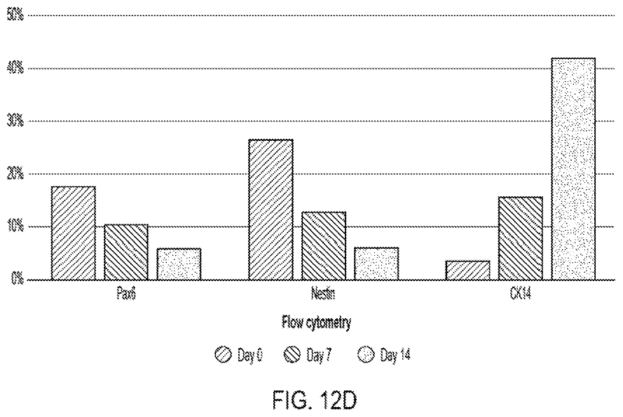

Protein expression levels of mature ectodermal cell markers were assessed after two weeks culturing single cell preparations of fresh ATZ crypts and anal skin in keratinocyte differentiation medium (KSFM, Sigma-Aldrich, St, Louis, Missouri). Cell clusters developed at day 3 and a cobblestone-like adherent layer with keratinocyte morphology formed in anal skin and ATZ cell cultures by day 10 (FIG. 12(a)). Co-expression of the keratinocyte markers, CK14 and CK15, were detected by immunostaining of anal skin adherent cultured cells (FIG. 12(b)) and ATZ adherent cultured cells (FIG. 12(c)).

Flow cytometric analysis of ATZ adherent culture cells showed that the ectodermal lineage marker, PAX6, expression decreased in culture from 18% at day 0 to 6% at day 7 and 5% at day 14 (FIG. 12(d)). CK14 expression on ATZ adherent culture cells increased over time in culture from 3% on day 0 to 18% on day 7 and 42% on day 14 (FIG. 12(d)).

Taken together these results support that fresh ATZ crypts have the capacity to generate mature cell types derived from the ectoderm lineage.

### Example 8. Absence of Multipotential Stem Cells of all Three Developmental Lineages in Porcine Small Intestine Crypts

This example demonstrates that porcine small intestine crypts have the potential to generate mature cells of the endodermal lineage, as expected but not mesodermal, and ectodermal lineages in vitro using the same methods as described in EXAMPLE 7 for porcine ATZ crypts for the purpose of describing and illustrating certain examples and embodiments of the present disclosure.

Small intestine single crypt cells were cultured for two weeks to promote differentiation of endoderm (human IntestiCult™), mesoderm (MethoCult™) and ectoderm (KSFM, Sigma-Aldrich).

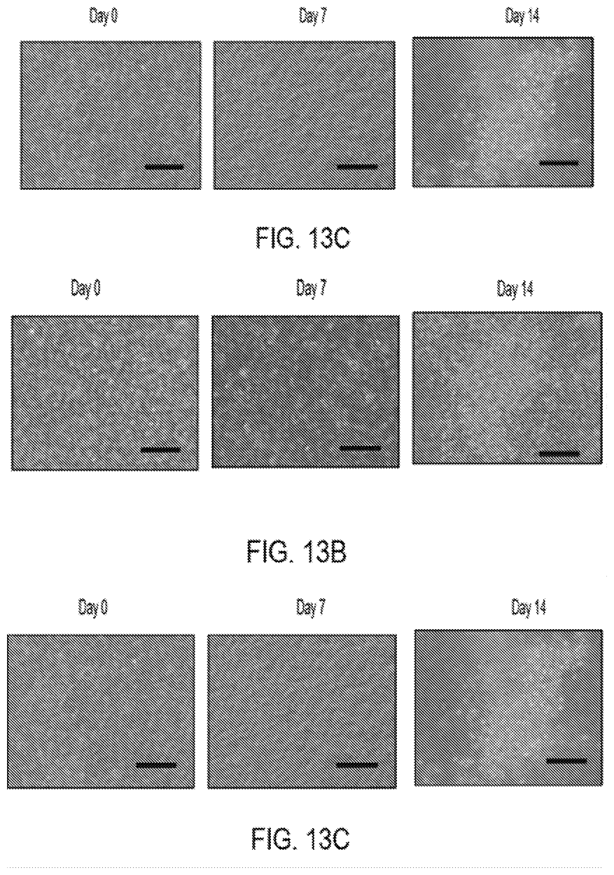

Small intestine crypt cells generated organoids (IntestiCult™), as expected (FIG. 13(a)). However, no growth was detected for small intestine crypt cells at days 7 or 14 when cultured in MethoCult™ (FIG. 13(b)) or KFSM (FIG. 13(c)).

Taken together, these results are consistent with the absence of multipotential stem cells in porcine small intestine crypts capable of generating cell types of all three developmental lineages, using the assays demonstrated for ATZ crypt cells.

### Example 9. In Vitro Embryoid Body Assay to Assess the Pluripotency of Porcine ATZ Crypt Cells

This example demonstrates that single cell preparations of fresh ATZ crypts cultured in feeder-free mTeSR™ medium can promote embryoid body formation for the purpose of describing and illustrating certain examples and embodiments of the present disclosure.

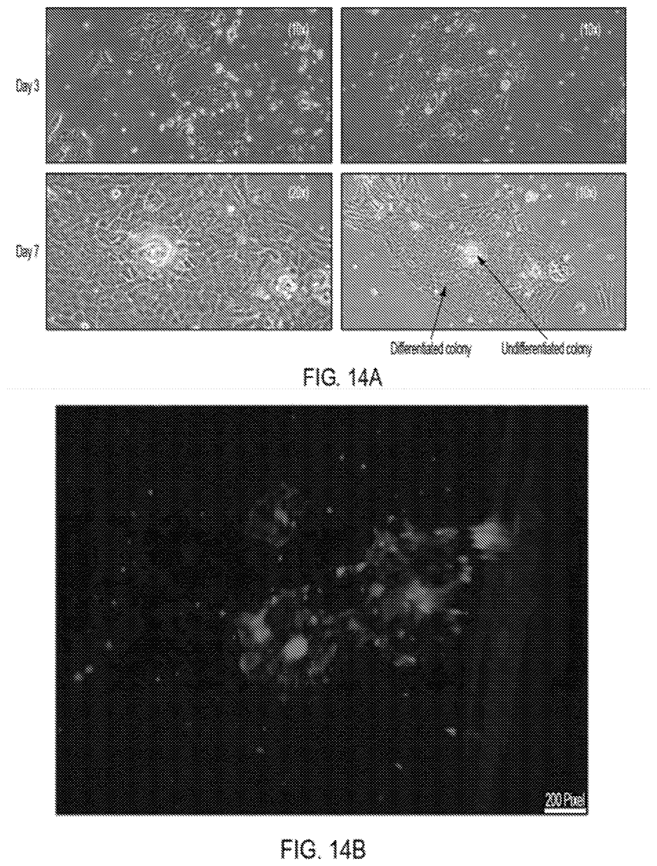

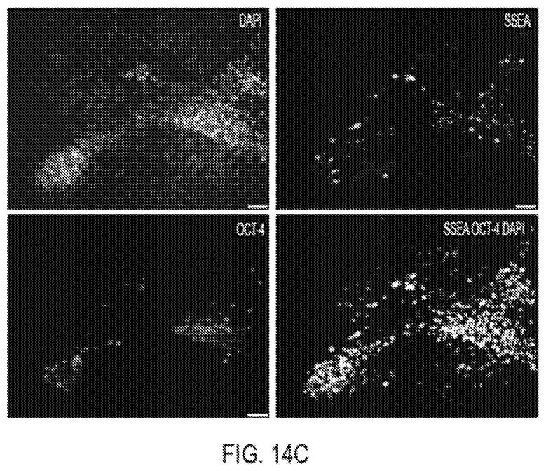

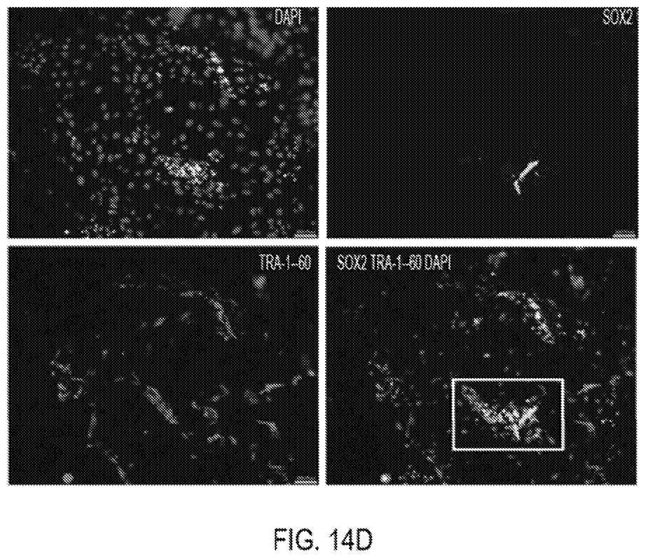

Fresh ATZ crypt single cells plated at a high density generated a cobblestone-like adherent layer by day 3; colonies of undifferentiated adherent cells developed by day 7; and differentiated adherent cells were generated and migrated away from the undifferentiated cell colonies (FIG. 14(a)).

Visualisation of alkaline phosphatase staining confirmed stem cell-like properties consistent with embryoid bodies (FIG. 14(b)). Furthermore, pluripotent stem cell markers, OCT4 and SSEA4 (FIG. 14(c)) and SOX2 and TRA-1-60 (FIG. 14(d)) were detected by immunocytochemistry at day 5.

Taken together, the results of the in vitro embryoid body assay are consistent with ATZ crypt cells exhibiting pluripotent stem cell-like properties.

### Example 10. Isolation of Crypts from Porcine Pancreatic Tissue

This example describes protocols for isolating ducts from pancreatic tissue.

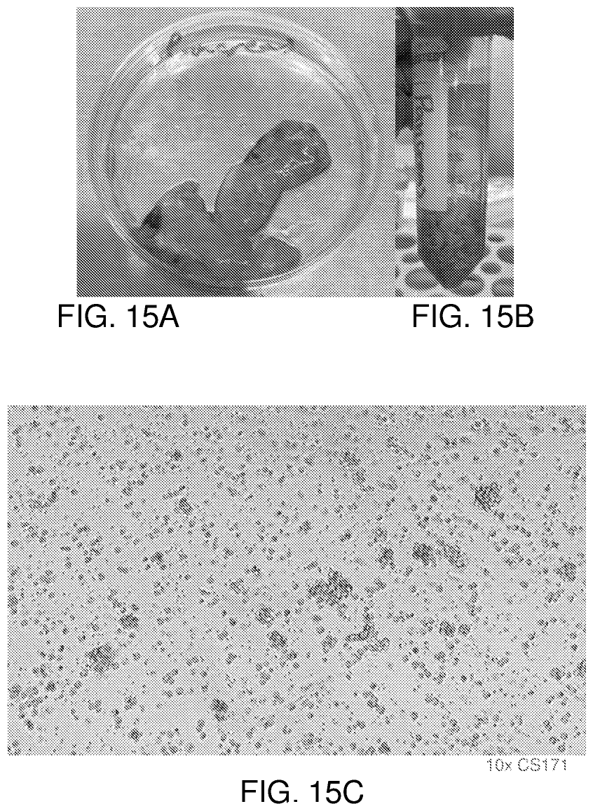

An example of healthy adult white Landcross porcine tissue from the pancreas was collected in AIMV medium (Thermo Fisher Scientific) containing antibiotics/antimycotics (AA) within 2 hours of termination and processed after overnight storage at 4° C. (FIG. 15(a)). The tissue was diced into 1 cm pieces and transferred to a 50 ml conical tube containing PBS+AA+Nystatin solution (FIG. 15(b)). The tissue was washed repeatedly until the PBS solution was clear of particulates. Enzymatic treatment of pancreatic tissue released ducts (FIG. 15(c)).

### Example 11. Establishment and Maintenance of Organoids Derived from Porcine ATZ and Pancreatic Tissues Cultured in PancreaCult™

This example describes how organoids derived porcine ATZ crypts in EXAMPLE 1 and pancreatic ducts in EXAMPLE 10 were cultured PancreaCult™ to promote pancreatic ductal organoid development and maintenance for the purpose of describing and illustrating certain examples and embodiments of the present disclosure.

ATZ and pancreatic crypts were separately embedded in Corning® Matrigel® Matrix with PancreaCult™.

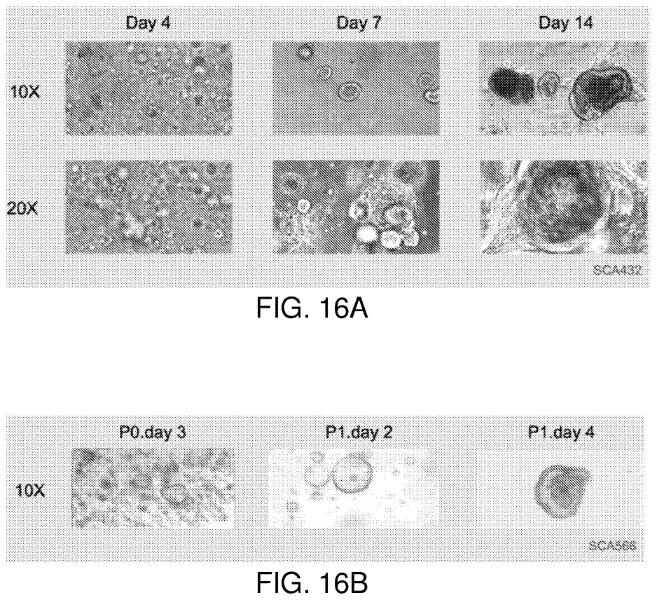

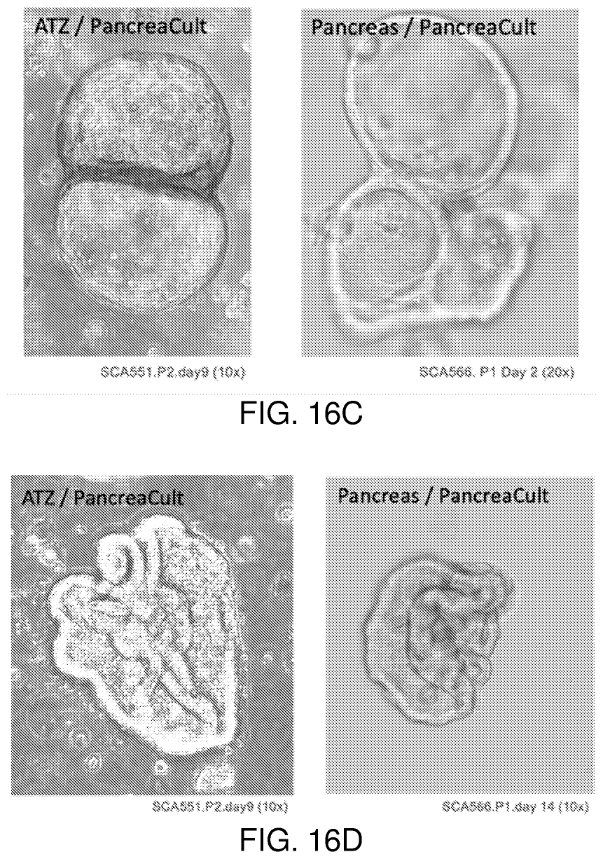

Small structures formed from ATZ crypts at day 4, filled in ring structures at day 7, and developed organoids at day 14 (FIG. 16(a)). Organoid structures formed from pancreatic ducts at day 3 and after passage at days 2 and 4 (FIG. 16(b)).

Although the rates of growth and development differed between ATZ and pancreatic organoids cultured in PancreaCult™, the outer rim and differentiated inner parts of the two organoid structures appeared similar (FIG. 16(c)).

Taken together these experimental results indicate that ATZ crypt cells differentiated into pancreatic organoids in vitro.

### Example 12. Protein Expression of Markers During Development of Pancreatic Organoids Derived from Porcine ATZ Crypts

This example shows protein expression of markers detected by immunofluorescence during development of porcine ATZ-derived pancreatic organoids generated in EXAMPLE 11 for the purpose of describing and illustrating certain examples and embodiments of the present disclosure.

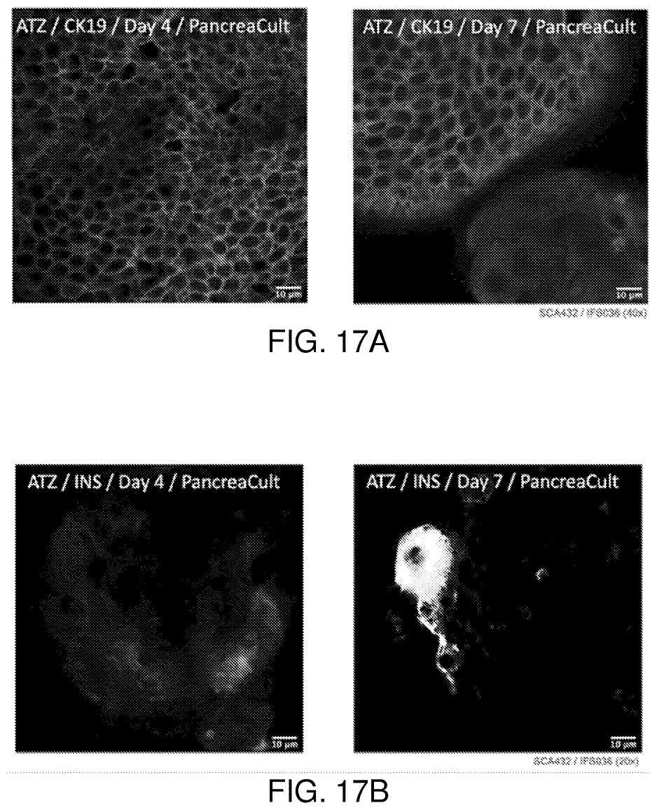

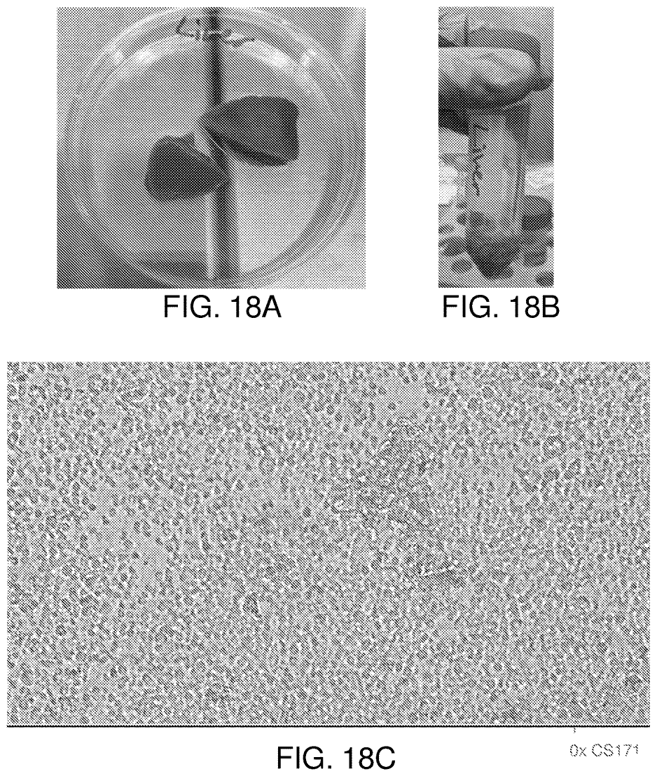

FIG. 17 shows markers detected by immunofluorescence of ATZ crypt-derived organoids grown in PancreaCult™ at days 4 and 7: (a) cytokeratin 19 (CK19), (b) insulin (INS).

### Example 13. MRNA Expression Profiling Markers Expressed During Development of Porcine ATZ Crypt-Derived Pancreatic Organoids

This example shows mRNA expression of markers detected by gradient qRT-PCR during the development of pancreatic organoids generated in EXAMPLE 11 for the purpose of describing and illustrating certain examples and embodiments of the present disclosure.

Transcriptional profiling of organoids derived from porcine ATZ crypt cells cultured in PancreaCult™ showed genes for the master regulators of pancreatic stem cell and progenitor cell development and tissue specific marker genes for acinar, alpha cells, beta cells and epithelial cells (TABLE 1).

TABLE 1

| Gene | Cells | Description |
|---|---|---|
| AMY | Acinar | Amylase is a digestive protein enzyme |
| | | produced by pancreatic acinar cells and |
| | | expressed at high levels |
| CK19 | Epithelial | Cytokeratins are intermediate filament |
| | | proteins responsible for the structural |
| | | integrity of epithelial cells |
| GCG | Alpha and L cells | Proglucagon is generated by alpha cells |
| | | in the pancreas and secreted to the |
| | | intestine in the distal ileum and colon |
| INS | Beta cells | Insulin is a peptide hormone produced |
| | | by beta cells of the pancreatic islets that |
| | | is encoded in humans by the INS gene |
| NKX6.1 | Beta cells | Homeobox protein Nkx-6.1 is required |
| | | for the development of pancreatic beta |
| | | cells |
| SOX9 | Master regulator | Transcription factor SOX-9 is a master |
| | | regulator of the pancreatic program |

The results of transcriptional profiling of pancreatic organoids derived from ATZ crypt cells for CK19 and INS were confirmed immunofluorescence results described in EXAMPLE 12.

Taken together, the PCR and immunofluorescence experimental results indicate that ATZ crypt cells differentiated into pancreatic cells capable of producing insulin in vitro.

### Example 14. Isolation of Stem Cells from Porcine Liver Tissue

This example describes protocols for isolating stem cells from liver tissue.

An example of healthy adult white Landcross porcine tissue from the liver was collected in AIMV medium (Thermo Fisher Scientific) containing antibiotics/antimycotics (AA) within 2 hours of termination and processed after overnight storage at 4° C. (FIG. 18(a)). The tissue was diced into 1 cm pieces and transferred to a 50 ml conical tube containing PBS+AA+Nystatin solution (FIG. 15(b)). The tissue was washed repeatedly until the PBS solution was clear of particulates. Enzymatic treatment of liver tissue released crypts (FIG. 18(c)).

### Example 15. Establishment and Maintenance of Organoids Derived from Porcine ATZ Crypts and Liver Tissues Cultured in HepatiCult™

This example describes how organoids derived porcine ATZ crypts in EXAMPLE 1 and liver stem cells in EXAMPLE 14 were cultured in HepatiCult™ to promote liver organoid development and maintenance for the purpose of describing and illustrating certain examples and embodiments of the present disclosure.

ATZ crypts and liver stem cells were separately embedded in Corning® Matrigel® Matrix with HepatiCult™.

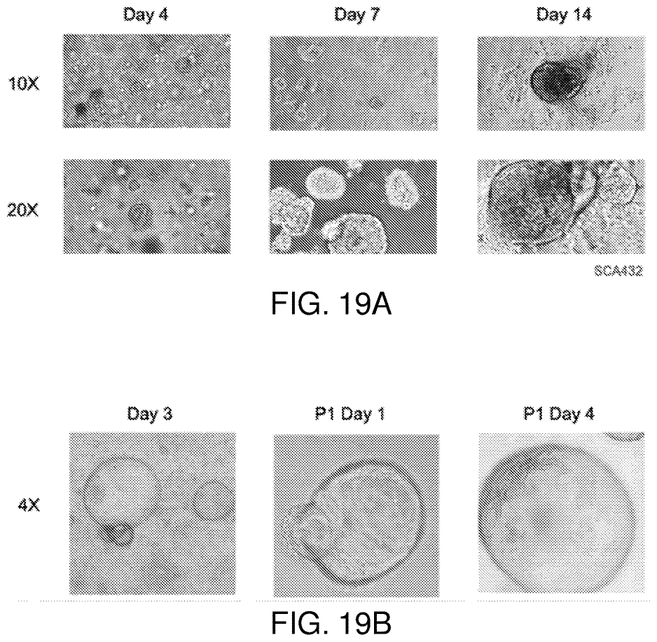

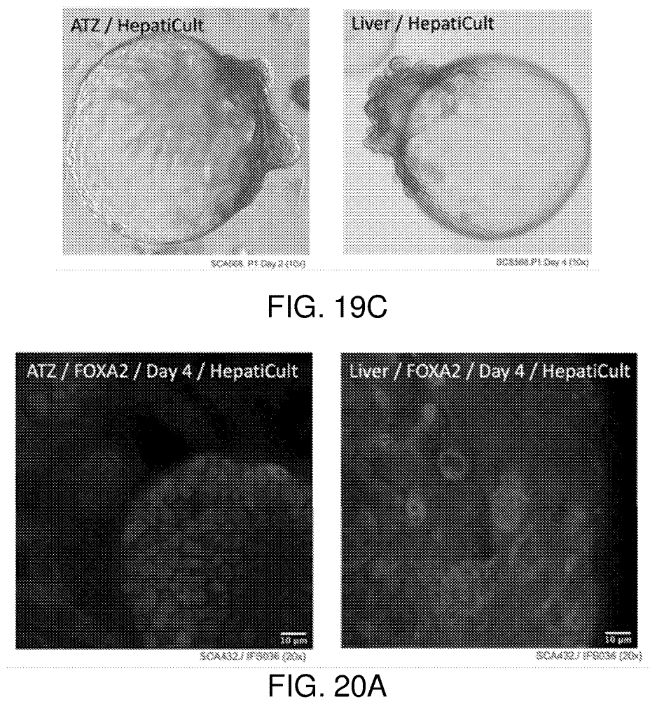

Small ring structures formed from ATZ-derived crypts at day 4, filled in ring structures at day 7, and developed organoids at day 14 (FIG. 19(a)). Small cystic organoid structures formed from liver-derived crypts at day 3, larger cystic organoid structures formed at days 1 and 4 after passage (FIG. 19(b)). Similar cystic organoid structures with a one-sided budding outgrowth were observed for ATZ crypts and liver stem cells grown in HepatiCult™ (FIG. 19(c)).

Taken together these experimental results indicate that ATZ crypt cells differentiated into liver cells in vitro.

### Example 16. Protein Expression of Markers During Development of Liver Organoids Derived from Porcine ATZ Crypts

This example shows protein expression of markers detected by immunofluorescence during development of porcine ATZ-derived liver organoids generated in EXAMPLE 15 for the purpose of describing and illustrating certain examples and embodiments of the present disclosure.

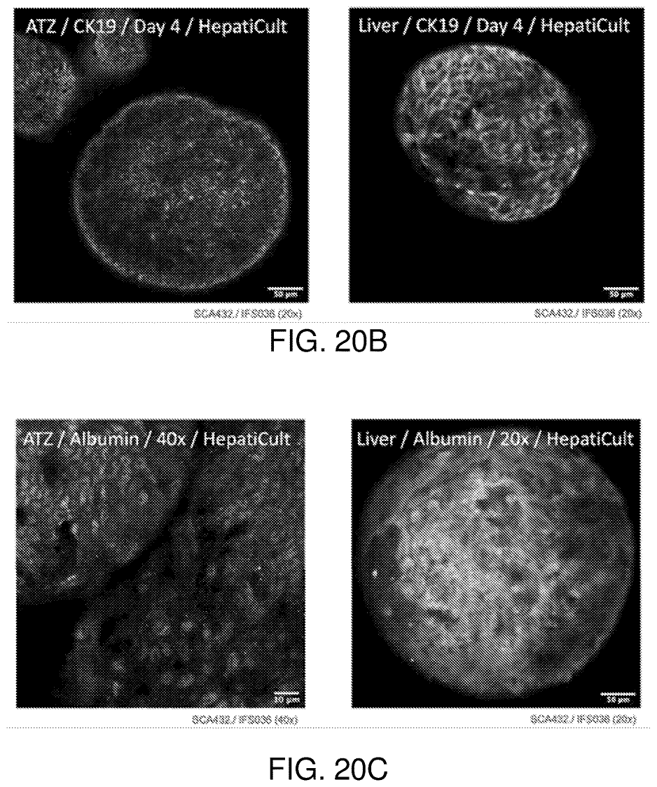

FIG. 20 shows protein markers detected by immunofluorescence of ATZ crypt and liver organoids grown in HepatiCult™: (a) FOXA2, (b) CK19, (c) ALB.

### Example 17. MRNA Expression Profiling Markers Expressed During Development of Porcine ATZ Crypt-Derived Liver Organoids

This example shows mRNA expression of markers detected by PCR during development of liver organoids generated in EXAMPLE 15 for the purpose of describing and illustrating certain examples and embodiments of the present disclosure.

Transcriptional profiling of organoids derived from porcine ATZ crypt cells cultured in HepatiCult™ showed genes for the master regulators of liver stem cell and progenitor cell development: FOXA2, SOX9, EPCAM (TABLE 2).

The results of transcriptional profiling of liver organoids derived from ATZ crypt cells for FOXA2 confirms immunofluorescence results described in EXAMPLE 15.

TABLE 2

| Gene | Description |
|---|---|
| SOX9 | Transcription factor SOX- is a master regulator of the |
| | pancreatic program |
| AFP | Alpha-fetoglobulin is a major plasma protein produced by the |
| | foetal liver during foetal development |
| FOXA2 | Hepatocyte nuclear factors are transcriptional activators for |
| | liver-specific genes such as albumin and transthyretin |
| HNF4A | The encoded protein controls the expression of several |
| | genes, including hepatocyte nuclear factor 1 alpha, a |
| | transcription factor which regulates the expression of several |
| | hepatic genes |
| ALB | This protein is secreted by hepatic beta cells and regulates |
| | blood plasma colloid osmotic pressure and acts as a carrier |
| | protein for a wide range of endogenous molecules including |
| | hormones, fatty acids, and metabolites, as well as exogenous |
| | drugs |
| CK19 | The protein encoded by this gene is a member of the keratin |
| | family. The keratins are intermediate filament proteins |
| | responsible for the structural integrity of epithelial cells |
| EPCAM | This gene encodes a carcinoma-associated antigen and is a |
| | member of a family that includes at least two type I |
| | membrane proteins. This antigen is expressed on most |
| | normal epithelial cells |

### Example 18. Isolation of Stem Cells from Porcine Lung Tissue

This example describes protocols for isolating stem cells from lung tissue.

An example of healthy adult white Landcross porcine tissue from the lung is collected in AIMV medium (Thermo Fisher Scientific) containing antibiotics/antimycotics (AA) within 2 hours of termination and processed after overnight storage at 4° C. Lung tissue is diced into 1 cm pieces and transferred to a 50 ml conical tube containing PBS+AA+Nystatin solution. The tissue is washed repeatedly until the PBS solution is clear of particulates and enzymatically treated to release single cells that include stem cells and progenitors.

### Example 19. Establishment and Maintenance of Organoids Derived from Porcine ATZ Crypts and Lung Tissues Cultured in Lung LSM

This example describes how porcine ATZ crypts isolated in EXAMPLE 1 and lung stem cells (bronchioalveolar stem cells and basal cells of the airway epithelium) in EXAMPLE 18 are cultured in lung LSM (e.g., STEMdiff™ Branching Lung Organoid Kit, or PneumaCult™ Airway Organoid Kit, or PneumaCult™ Apical-Out Airway Organoid Medium, or PneumaCult™ Alveolar Organoid Medium, all from STEMCELL Technologies, Inc; Human Lung Organoid Culture Protocols, Bio-Techne;

3dGRO™ Lung Organoid Branching Medium3dGRO™ and Lung Organoid Maturation Medium, Merck) to promote lung organoid development and maintenance for the purpose of describing and illustrating certain examples and embodiments of the present disclosure.

ATZ crypts and lung stem cells are separately embedded in Corning® Matrigel® Matrix with lung LSM.

### Example 20. MRNA Expression Profiling Markers Expressed During Development of Porcine ATZ Crypt-Derived Lung Organoids

This example shows mRNA expression of markers detected by PCR during development of lung organoids generated from porcine ATZ crypts and lung tissue in EXAMPLE 19 for the purpose of describing and illustrating certain examples and embodiments of the present disclosure.

Transcriptional profiling of organoids derived from porcine ATZ crypt cells that are cultured in lung LSM show genes for the master regulators of lung stem cell and progenitor cell development and proximal and distal-like branching airway epithelial structures expressing a group of markers EPCAM, MUC1, NKX2.1, P63, SOX2, or SOX9. When maintained beyond 28 days in culture, there is an increase in expression levels of markers for more mature lung cells, such as ABCA3, SFTPB, and SFTPC.

### Example 21. Isolation of Stem Cells from Porcine Thyroid Tissue

This example describes protocols for isolating stem cells from thyroid tissue.

An example of healthy adult white Landcross porcine tissue from the thyroid is collected in AIMV medium (Thermo Fisher Scientific) containing antibiotics/antimycotics (AA) within 2 hours of termination and processed after overnight storage at 4° C. thyroid tissue is diced into 1 cm pieces and transferred to a 50 ml conical tube containing PBS+AA+Nystatin solution. The tissue is washed repeatedly until the PBS solution is clear of particulates and enzymatically treated to release single cells that include stem cells and progenitors.

### Example 22. Establishment and Maintenance of Organoids Derived from Porcine ATZ Crypts and Thyroid Tissues Cultured in Thyroid LSM

This example describes how porcine ATZ crypts isolated in EXAMPLE 1 and thyroid stem cells in EXAMPLE 22 are cultured in thyroid LSM (e.g., as described in protocols “Adult mouse and human organoids derived from thyroid follicular cells and modelling of Graves' hyperthyroidism”, PNAS, 118(51): e1-11)) to promote thyroid organoid development and maintenance for the purpose of describing and illustrating certain examples and embodiments of the present disclosure.

ATZ crypts and thyroid stem cells are separately embedded in Corning® Matrigel® Matrix with thyroid LSM.

### Example 23. MRNA Expression Profiling Markers Expressed During Development of Porcine ATZ Crypt-Derived Thyroid Organoids

Transcriptional profiling of organoids derived from porcine ATZ crypt cells that are cultured in thyroid LSM show genes for the master regulators of lung stem cell and progenitor cell development.

At day 28, increased in expression levels of markers for more mature thyroid cells (e.g., PAX8 and NKX2.1) is observed.

### Example 24. Isolation of Intestinal Crypts from Anorectal Tissue of an Idiopathic Perianal Fistula Patient

This example describes a protocol for isolating intestinal crypts from anorectal tissue of a human subject with an idiotypic perianal fistula.

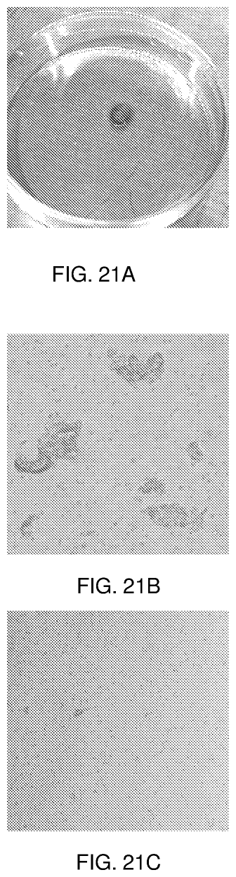

A biopsy from anorectal tissue of an idiopathic perianal fistula patient was collected in AIMV V medium containing antibiotics/antimycotics within 2 hours of termination and processed directly, or in some instances after overnight storage in at 4° C. (FIG. 21(a)). Enzymatic treatment of minced ATZ tissue released intestinal crypts (FIG. 21(b)) and single cell preparations were prepared (FIG. 21(c)).

### Example 25. MRNA Expression Profiling by qPCR of Freshly Isolated ATZ Crypt Cells from an Idiopathic Perianal Patient

This example shows mRNA expression profiling by qPCR of idiopathic perianal patient ATZ crypt cells for the purpose of describing and illustrating certain examples and embodiments of the present disclosure.

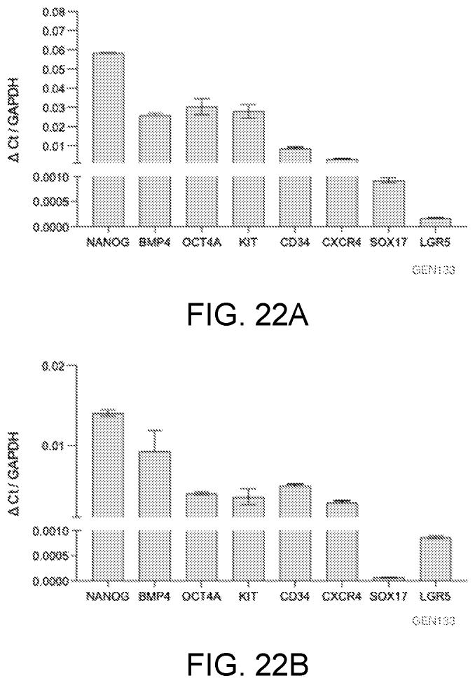

Transcriptional profiling of freshly isolated ATZ crypts from two idiopathic perianal patients expressed markers for pluripotent stem cells (NANOG, OCT4A) and multipotent stem cells and progenitor cells (BMP4, KIT, CD34, CXCR4, LGR5) and developmental stem cells of the endoderm (SOX17) (FIG. 22(a-b)).

Taken together with mRNA profiling of porcine ATZ crypts in EXAMPLE 5, these results confirm that ATZ crypts contain populations of pluripotent and multipotent stem cells.

### Example 26. Crypt Anorectal Transition Zone Organoid Development from an Idiopathic Perianal Fistula Patient

Crypt ATZ organoid development from human tissue of EXAMPLE 24 was developed.

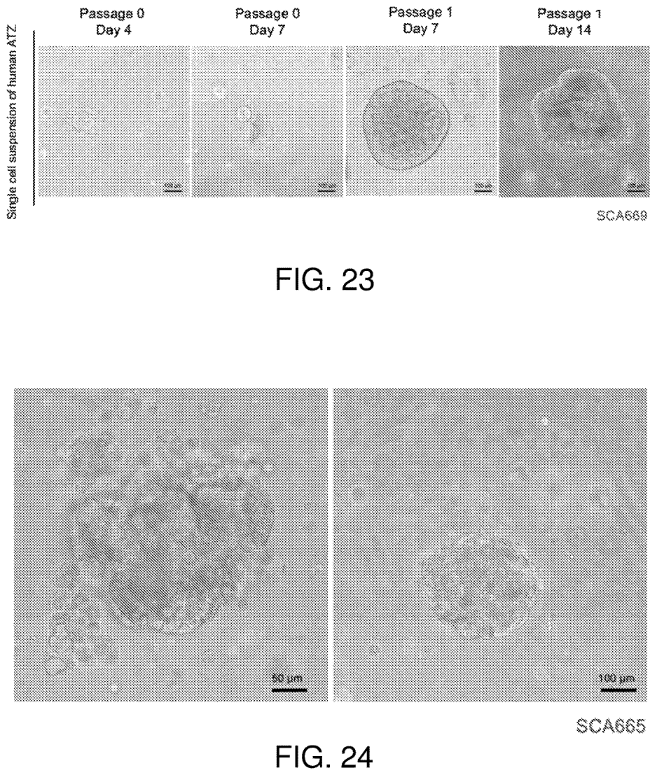

Crypts or single cell preparations of ATZ tissue prepared in EXAMPLE 24 were embedded in Corning® Matrigel® Matrix with human IntestiCult™ and developed into fully formed organoids in 1-3 weeks (FIG. 23). Clusters of cells formed on day 4 and organized structures formed on day 7 (both at passage 0); compact, thickened organoids formed on day 7 and 14 of passage 1.

### Example 27. Establishment and Maintenance of Organoids Derived from Idiopathic Perianal Fistula Patient ATZ Tissue Cultured in PancreaCult™

This example describes how organoids derived idiopathic perianal fistula patient ATZ crypts or single cell preparations in EXAMPLE 24 were cultured PancreaCult™ to promote pancreatic organoid development and maintenance for the purpose of describing and illustrating certain examples and embodiments of the present disclosure.

ATZ crypts and single cells were embedded in Corning® Matrigel® Matrix with PancreaCult™. Organised, complex organoids formed after 1 passage (FIG. 24).

These results, taken together pancreas organoid establishment and maintenance from porcine ATZ crypts and single cells prepared in EXAMPLE 11, confirm that ATZ crypts have the capacity to differentiate into liver organoids in vitro.

### Example 28. Implantation and Recovery Donor Porcine ATZ Crypt Cells Delivered in a Collagen Paste to the Pancreas of a Recipient Pig to Test Engraftment

This example describes how single cell preparations of porcine male ATZ crypts in EXAMPLE 1 were implanted in the pancreas of a healthy female pig to test engraftment and cellular expansion for the purpose of describing and illustrating certain examples and embodiments of the present disclosure.

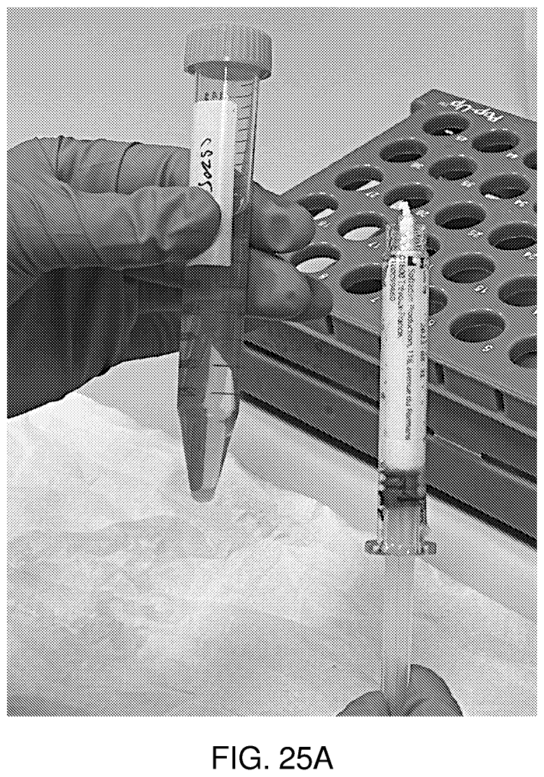

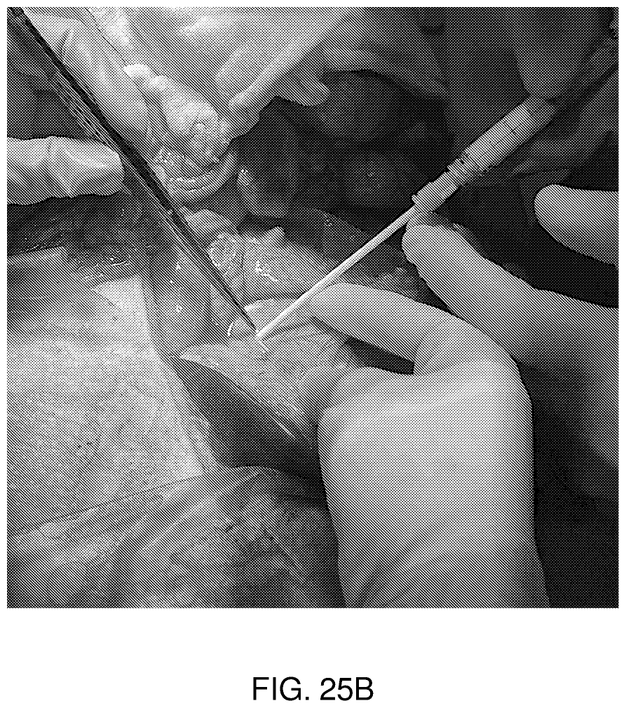

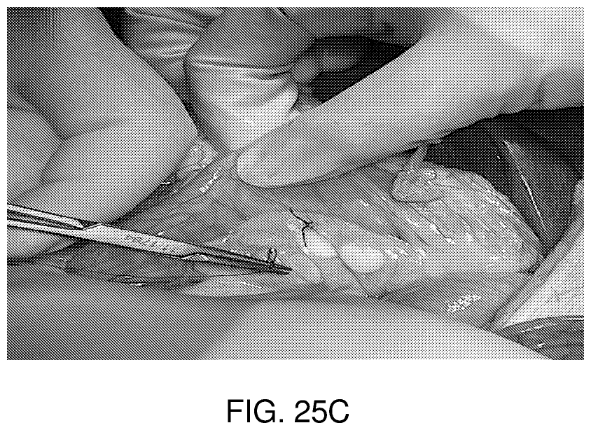

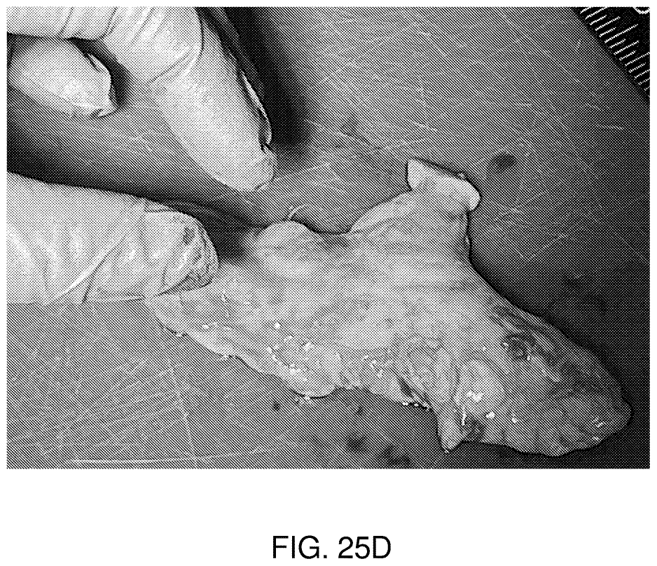

Single cell preparations of cryopreserved male porcine crypts in EXAMPLE 1 were thawed, resuspended in AIM V medium, centrifuged, and cell pellets were mixed with a collagen paste (Permacol™) in a syringe (FIG. 25(a)). An Abbocath Catheter 14F was attached to the syringe containing ATZ cells in Permacol™, mixed well, injected into three places in the pancreas (ATZ/Permacol™ implant) (FIG. 25(b)); secured by sutures (FIG. 25(c)). After 5 weeks, the pig was sacrificed, and the ATZ/Permacol implant and control pancreas tissue were surgically resected (FIG. 25(d)). The implantation sites were still visible (indicated by arrows in the image). The tissue was sectioned; half was placed in neutral buffered formalin (NBF) for histology and the other half in RNAlater reagent (Thermo Fisher) for mRNA profiling.

The ATZ cell-transplanted pig showed no evidence of inflammation nor transplant rejection after 5 weeks.

### Example 29. Histologic Staining of Pancreatic Tissue Recovered 5 Weeks after Implantation of Donor Porcine ATZ Crypt to the Pancreas of a Recipient Pig

This example shows the presence of porcine male donor ATZ cell engraftment in the pancreas of a healthy pig after five weeks for the purpose of describing and illustrating certain examples and embodiments of the present disclosure.

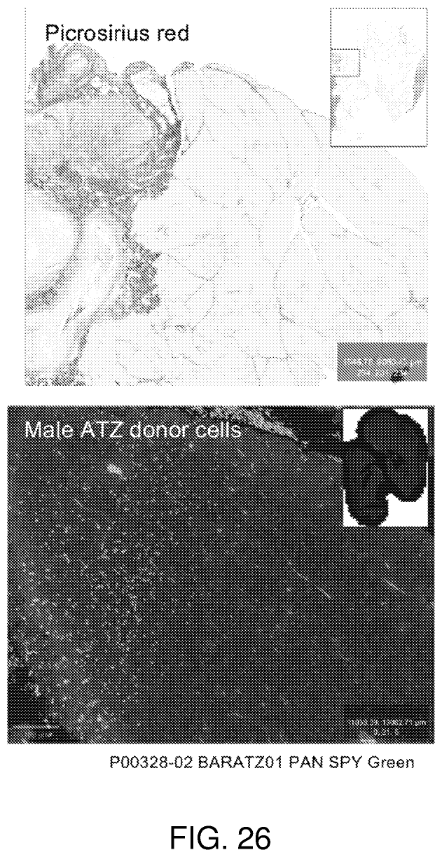

Histologic staining of the ATZ cell implanted section of the resected pancreas in EXAMPLE 29 (FIG. 26) showed collagen fibre staining detected by picrosirius red (dark gray) in the upper panel, consistent with a newly forming support matrix. The bottom panel shows fluorescence in situ hybridization (FISH) of the SRY gene, a sex determining gene on the Y chromosome, which was confirmed by detection of male DNA by PCR (not shown), indicating the presence of male ATZ donor cell migration in the recipient pig pancreas.

### Example 30. Molecular Profiling of Pancreatic Tissue Recovered 5 Weeks after Implantation of Donor Porcine ATZ Crypt Cells to the Pancreas of a Recipient Pig

This example shows the presence by molecular profiling of porcine male donor ATZ crypt cell engraftment and expansion in the pancreas of a healthy pig after five weeks for the purpose of describing and illustrating certain examples and embodiments of the present disclosure.

mRNA profiling by RNA Seq was performed on male porcine donor ATZ crypt cells cultured in IntestiCult™, cells harvested from the ATZ/Permacol™ implant in pancreas after 5 weeks, and control pancreatic tissue from the ATZ implantation study in EXAMPLE 28.

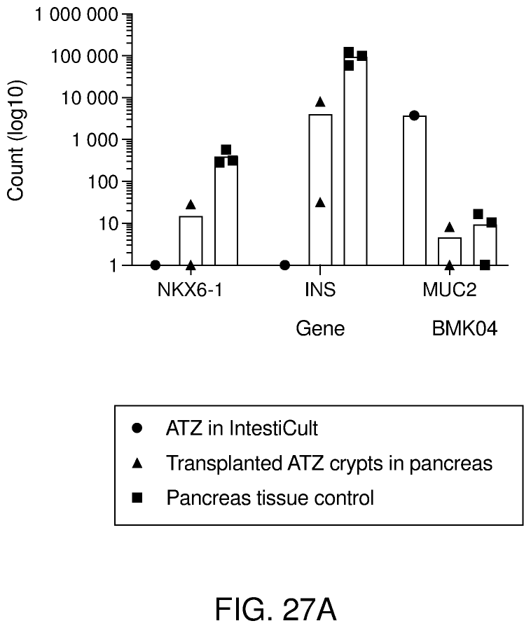

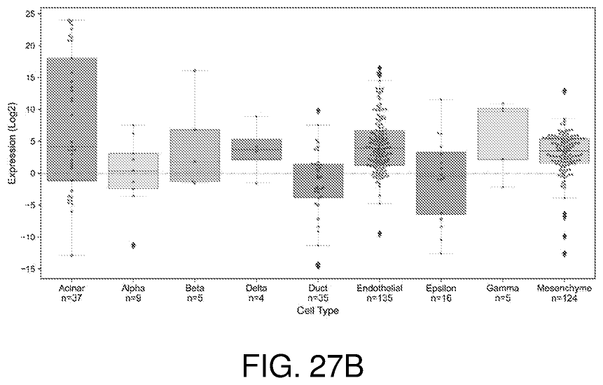

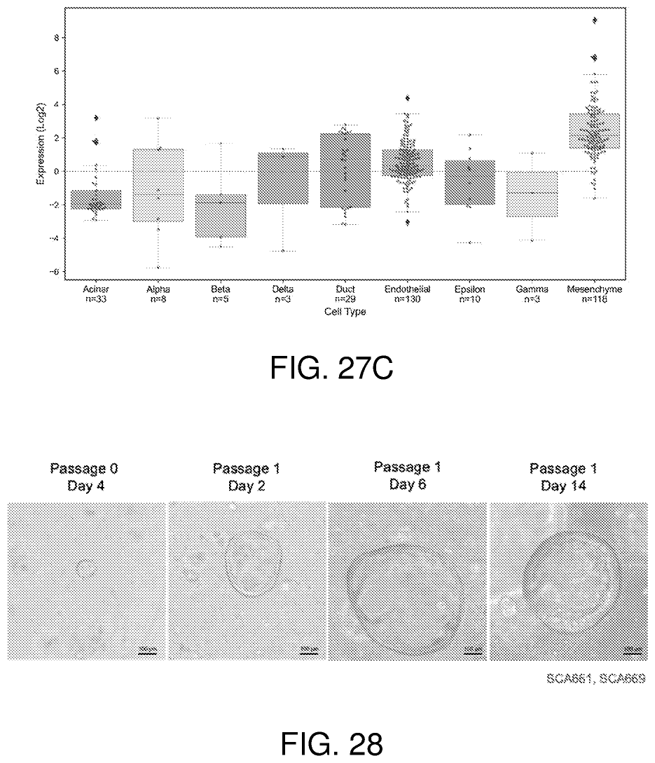

FIG. 27(a) show that genes associated with pancreas beta cell development (NKX6-1) and insulin (INS) production that were detected in the ATZ/Permacol™ implant and control pancreatic tissue but not in the ATZ donor cells cultured in IntestiCult™. As expected, expression of MUC-2, a gene encoding mucin, was detected in the ATZ donor cells cultured in IntestiCult™; only low levels of MUC-2 were detected in the ATZ/Permacol™ implant and control pancreatic tissues.

FIG. 27(b) shows differential gene expression profiling of human pancreas cell type specific marker genes (from Muraro M J et al. (2016) “A Single-Cell Transcriptome Atlas of the Human Pancreas.” Cell Syst. 3(4):385-394.e3) in the porcine ATZ/Permacol™ implant compared to the donor porcine ATZ cells cultured in IntestiCult™. These data show that the ATZ/Permacol™ implant expressed relatively increased mRNA levels of some pancreas-related cell-type marker genes (acinar, beta, and delta, epsilon cells) and increased levels of genes relating to tissue remodeling cells (endothelial and mesenchyme cells).

FIG. 27(c) shows differential gene expression profiling of human pancreas cell type specific marker genes (from Muraro M J et al. (2016) “A Single-Cell Transcriptome Atlas of the Human Pancreas.” Cell Syst. 3(4):385-394.e3) in the porcine ATZ/Permacol™ implant compared to the pancreatic control tissue. These data show that the ATZ/Permacol™ implant expressed relatively lower mRNA levels of some pancreas-related cells (acinar, alpha, beta, and gamma cells), as expected from early stage engraftment of ATZ donor cells, and increased levels of genes relating to tissue remodelling cells (endothelial and mesenchyme cells).

Taken together, these mRNA profiling results showed that the male porcine donor ATZ cells that differentiated into intestinal cells in vitro showed the capacity to form functional pancreatic cells in vivo, supporting the multipotential nature of ATZ cells, and that the ATZ/Permacol™ implant remodelled liver.

### Example 31. Establishment and Maintenance of Organoids Derived from Idiopathic Perianal Fistula Patient ATZ Crypts Cultured in HepatiCult™

This example describes how organoids derived from idiopathic perianal fistula patient ATZ crypts or single cell preparations in EXAMPLE 24 were cultured in HepatiCult™ to promote liver organoid development and maintenance for the purpose of describing and illustrating certain examples and embodiments of the present disclosure.

ATZ crypts or single cells were embedded in Corning® Matrigel® Matrix with HepatiCult™. A ring structure formed on day 4, larger cystic organoids at day 2 and day 6 after passage 1; and filled in, compact, thickened organoids formed by day 14 of passage 1 (FIG. 28(a-b)).

These results, taken together liver organoid establishment and maintenance from porcine ATZ crypts and single cells prepared in EXAMPLE 15, confirm that ATZ crypts have the capacity to differentiate into liver organoids in vitro.

### Example 32. Implantation and Recovery Donor Porcine ATZ Crypt Cells Delivered in a Collagen Paste to the Liver of a Recipient Pig to Test Engraftment

This example describes how single cell preparations of porcine male ATZ crypts in EXAMPLE 1 were implanted in the liver of a healthy female pig to test engraftment and cellular expansion for the purpose of describing and illustrating certain examples and embodiments of the present disclosure.

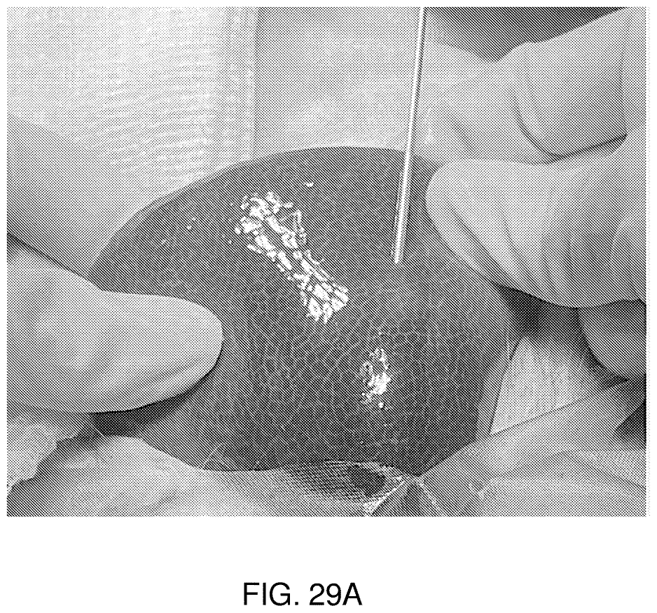

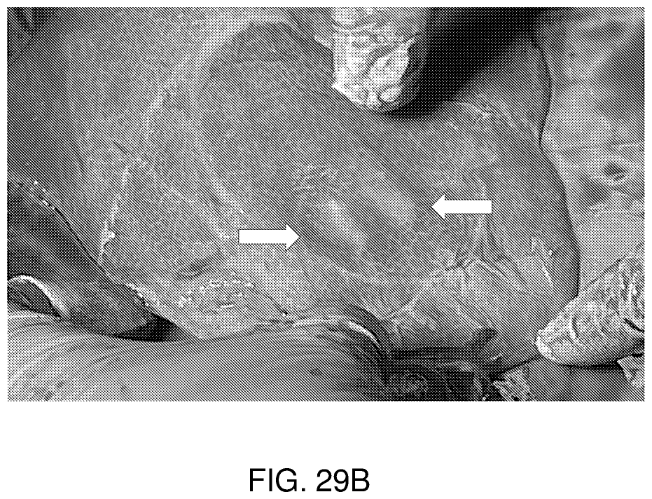

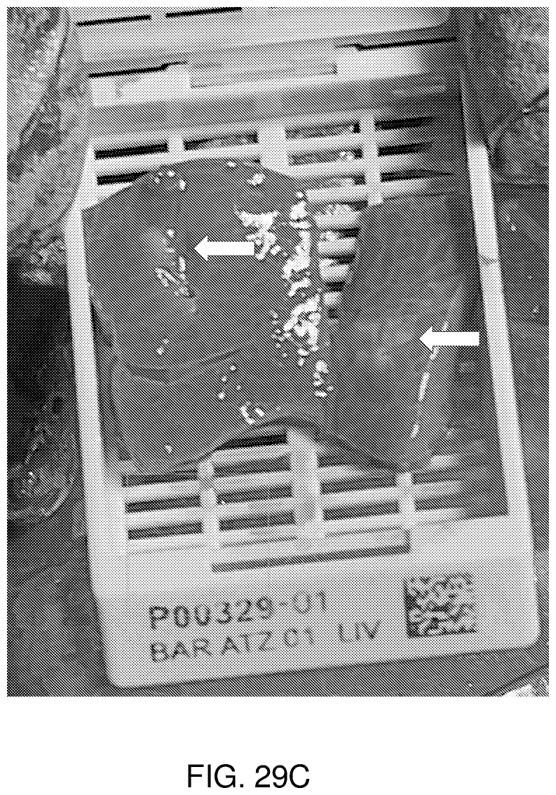

Single cell preparations of cryopreserved male porcine crypts in EXAMPLE 1 were thawed, resuspended in AIM V medium, centrifuged, and cell pellets were mixed with a collagen paste (Permacol™) in a syringe. A wide gauge needle was attached to the syringe containing ATZ cells in Permacol™, mixed well, injected into three places in the pancreas (ATZ/Permacol™ implant); each injection site was sutured (not shown) (FIG. 29(a)). After 5 weeks, the pig was sacrificed, and the ATZ/Permacol implant and liver control tissue were surgically resected (FIG. 29(b)). The resected tissue was sectioned; half was placed in neutral buffered formalin (NBF) for histology (FIG. 29(c)) and the other half in RNAlater reagent (Thermo Fisher) for mRNA profiling. The implantation sites were still visible (indicated by arrows in the image) in the resected tissues.

The ATZ cell-transplanted pig showed no evidence of inflammation nor transplant rejection after 5 weeks.

### Example 33. Histological Staining of Liver Tissue Recovered 5 Weeks after Implantation of Donor Porcine ATZ Crypt Cells to the Liver of a Recipient Pig

This example shows the presence of porcine male donor ATZ cell engraftment in the liver of a healthy pig after five weeks for the purpose of describing and illustrating certain examples and embodiments of the present disclosure.

Histologic staining of the ATZ/Permacol™ implant section of the resected liver in EXAMPLE 32.

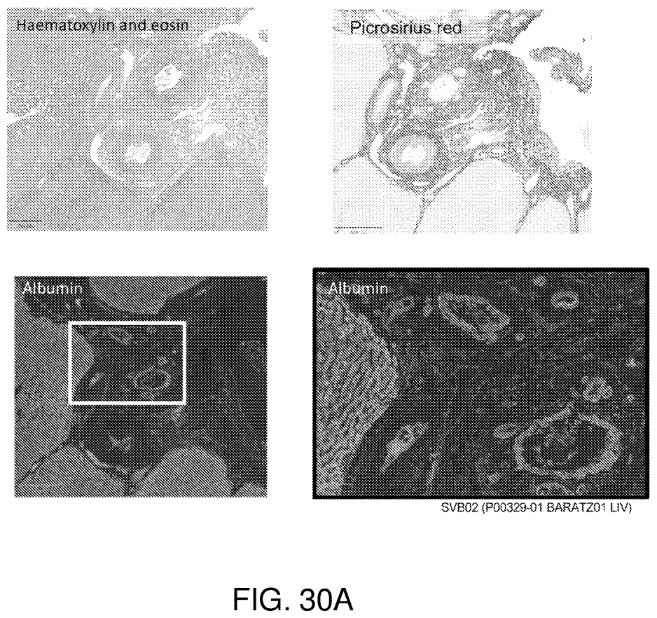

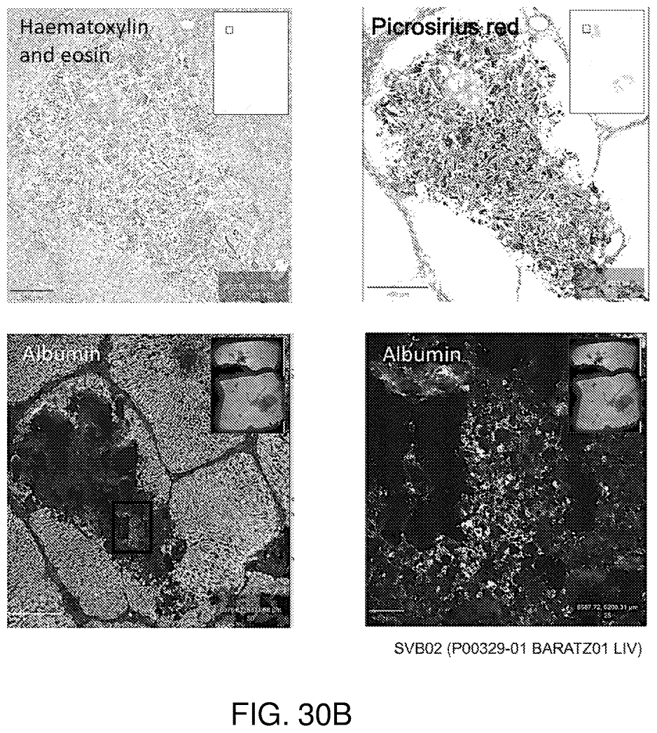

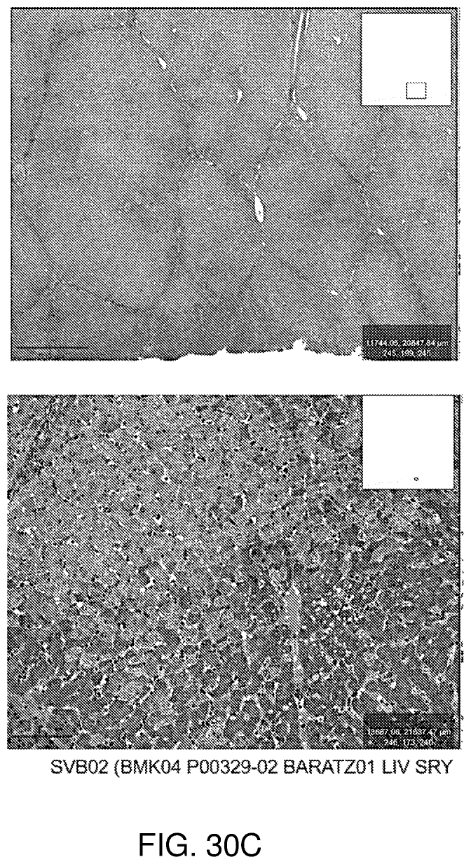

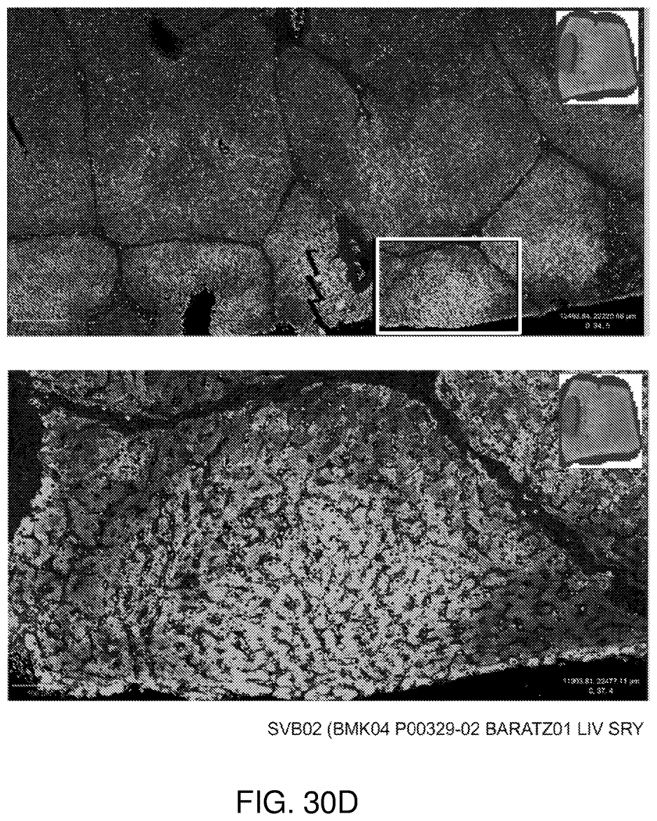

FIG. 30(a) shows a section of liver with newly forming complex structures of large and small vasculature or ducts visualised by staining: haematoxylin and eosin (top left panel), collagen fibre staining detected by picrosirius red (dark grey) in the upper right panel, and albumin (light grey) in the lower left panel and in the insert in the lower right panel.

FIG. 30(b) shows a section of liver stained with haematoxylin and eosin and picrosirius red in the upper panels. Within the ATZ/Permacol™ implant, a cluster of albumin producing cells was detected in the lower left panel and in the insert in the lower right panel.

FIG. 30(c) shows a section of liver distal from the ATZ/Permacol™ implant site stained with haematoxylin and eosin identifying apparent regenerating liver tissue in the upper panel and highlighted in the region of selected interest in the lower panel.

FIG. 30(d) show staining by FISH for the SRY male gene (light grey) highlighted in the region of selected interest in the lower panel. This figure demonstrates that male ATZ donor cells have the capacity to migrate from the ATZ/Permacol™ implant site, shown in the top right corner of the upper panel, and to generate new tissue. Further, the FISH SRY staining cells shows that the different cellular structure of the new tissue highlighted in FIG. 30(c) consists of the same cell type as the adjacent established liver.

Taken together FIG. 30(d) demonstrates that porcine donor ATZ crypt cells have a robust capacity to engraft, migrate and regenerate liver tissue in a recipient female pig.

### Example 34. Molecular Profiling of Pancreatic Tissue Recovered 5 Weeks after Implantation of Donor Porcine ATZ Crypt Cells to the Liver of a Recipient Pig

This example shows the presence by molecular profiling of porcine male donor ATZ crypt cell engraftment and the generation of new liver tissue in a healthy pig after five weeks for the purpose of describing and illustrating certain examples and embodiments of the present disclosure.

mRNA profiling by RNA Seq was performed on male donor ATZ crypt cells cultured in IntestiCult™, cells harvested from the ATZ/Permacol™ implant in liver after 5 weeks, and control pancreatic tissue from the ATZ implantation study in EXAMPLE 32.

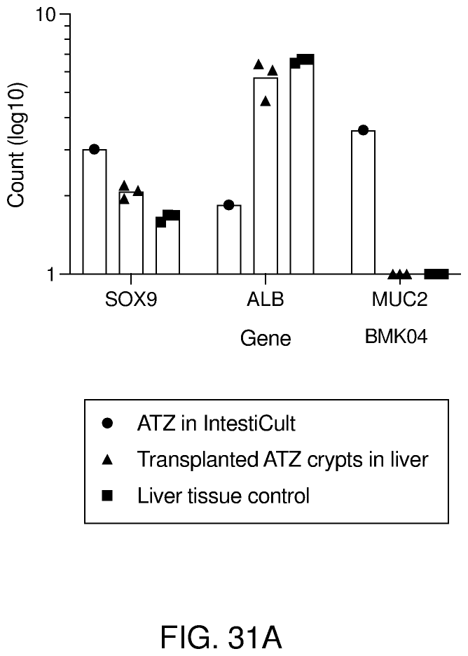

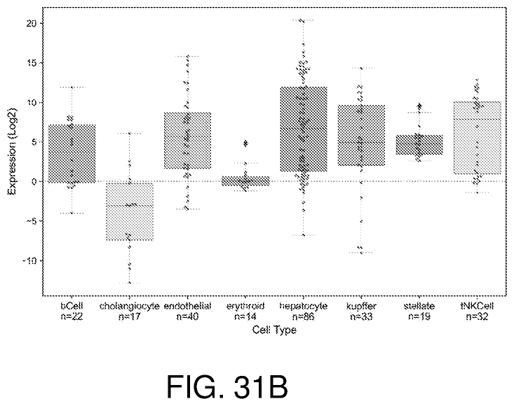

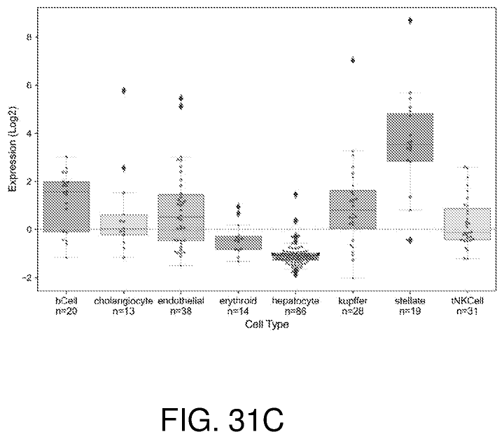

FIG. 31(a) shows that genes associated with liver beta cell development (NKX6-1) and insulin (INS) production were detected in the ATZ/Permacol™ implant and control pancreatic tissue but not in the ATZ donor crypt cells cultured in IntestiCult™. As expected, expression of MUC-2, a gene encoding mucin, was detected in the ATZ donor cells cultured in IntestiCult™; only low levels of MUC-2 were detected in the ATZ/Permacol™ implant and control liver tissues.

FIG. 31(b) shows differential gene expression profiling of human liver cell type specific marker genes (from MacParland, S A, et al. (2018) “Single cell RNA sequencing of human liver reveals distinct intrahepatic macrophage populations.” Nat Commun. 2018 Oct. 22; 9(1):4383) in the porcine ATZ/Permacol™ implant in liver compared to the donor porcine ATZ crypt cells cultured in IntestiCult™. These data show that the ATZ/Permacol™ implant expressed relatively increased mRNA levels of liver-related cell-type marker genes are increased.

FIG. 31(c) shows differential gene expression profiling of human pancreas cell type specific marker genes (from MacParland, S A, et al. (2018) “Single cell RNA sequencing of human liver reveals distinct intrahepatic macrophage populations.” Nat Commun. 2018 Oct. 22; 9(1):4383) in the porcine ATZ/Permacol™ implant compared to the liver control tissue. These data show that the ATZ/Permacol™ implant expressed relatively lower mRNA levels of hepatocytes are lower, as may be expected for early stage regeneration, and increased levels of genes relating to tissue remodelling cells (endothelial and stellate cells).

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

1. A cellular composition for use in cell transplantation to treat a damaged or dysfunctional endodermal tissue in a subject, comprising:
a mixed population of ATZ cells comprising multipotent ATZ stem cells and endodermal progenitor ATZ cells in a pharmaceutically-acceptable carrier or exogenous biocompatible scaffold.

2. The cellular composition of claim 1, wherein:
the multipotent ATZ stem cells comprise at least 5% of the total ATZ cells.

3. The cellular composition of claim 1 or claim 2, wherein:
the multipotent ATZ stem cells do not express significant levels of CD45.

4. A method of producing a cellular composition for use in cell transplantation to treat a damaged or dysfunctional endodermal tissue in a subject, comprising:
(a) obtaining a sample of ATZ cells that are autologous or allogeneic to the subject;
(b) isolating a mixed population of ATZ cells comprising multipotent ATZ stem cells and endodermal progenitor ATZ cells from the sample; and
(c) expanding the multipotent ATZ stem cells in a minimal growth medium (MGM).

5. The method of claim 4, wherein:
the multipotent ATZ stem cells do not express significant levels of CD45.

6. The method of any one of claims 4-5, wherein:
the multipotent ATZ stem cells express markers CD34, CD117 and CD184.

7. The method of any one of claims 4-6, wherein:
the multipotent ATZ stem cells express markers NANOG and OCT4A.

8. The method of any one of claims 4-7, wherein:
wherein the step of expanding the multipotent ATZ stem cells comprises replating the ATZ cells through at least 5 passages in MGM.

9. The method of any one of claims 4-8, further comprising:
(d) culturing the ATZ cells in an endodermal lineage-specific medium (LSM) to promote at least partial differentiation of the multipotent ATZ stem cells to produce an expanded population of endodermal progenitor ATZ cells.

10. The method of claim 9, wherein:
the damaged or dysfunctional endodermal tissue is pancreatic tissue and the LSM comprises a pancreatic-specific LSM.

11. The method of claim 10, wherein:
wherein the LSM promotes differentiation of multipotent ATZ stem cells to produce a population of ATZ stem cell-derived pancreatic organoid cells.

12. The method of claim 9, wherein:
the damaged or dysfunctional endodermal tissue is hepatic tissue and the LSM comprises a hepatic-specific LSM.

13. The method of claim 12, wherein:
wherein the LSM promotes differentiation of multipotent ATZ stem cells to produce a population of ATZ stem cell-derived hepatic organoid cells.

14. The method of claim 9, wherein:
the damaged or dysfunctional endodermal tissue is intestinal mucosa tissue and the LSM comprises an intestinal mucosa-specific LSM.

15. The method of claim 14, wherein:
wherein the LSM promotes differentiation of multipotent ATZ stem cells to produce a population of ATZ stem cell-derived intestinal mucosa organoid cells.

16. The method of claim 9, wherein:
the damaged or dysfunctional endodermal tissue is intestinal mucosa tissue and the LSM comprises an intestinal mucosa-specific LSM.

17. The method of claim 16, wherein:
wherein the LSM promotes differentiation of multipotent ATZ stem cells to produce a population of ATZ stem cell-derived intestinal mucosa organoid cells.

18. A method treating damaged or dysfunctional endodermal tissue in a subject, comprising:
(a) obtaining a sample of ATZ cells that are autologous or allogeneic to the subject;
(b) isolating a mixed population of ATZ cells comprising multipotent ATZ stem cells and endodermal progenitor ATZ cells from the sample;
(c) expanding the multipotent ATZ stem cells within the mixed population in a minimal growth medium (MGM), and.
(d) introducing a therapeutically effective amount of the multipotent ATZ stem cells within the mixed population into the damaged or dysfunctional tissue in the subject.

19. A method treating damaged or dysfunctional endodermal tissue in a subject, comprising:
(a) obtaining a sample of ATZ cells that are autologous or allogeneic to the subject;
(b) isolating a mixed population of ATZ cells comprising multipotent ATZ stem cells and endodermal progenitor ATZ cells from the sample;
(c) expanding the multipotent ATZ stem cells within the mixed population by culturing the mixed population in a minimal growth medium (MGM),
(d) culturing the mixed population in an endodermal lineage-specific medium (LSM) to promote at least partial differentiation of multipotent ATZ stem cells within the mixed population to produce an expanded population of endodermal progenitor ATZ cells within the mixed population; and
(e) introducing a therapeutically effective amount of the multipotent ATZ stem cells and endodermal progenitor ATZ cells into the damaged or dysfunctional tissue in the subject.

20. The method of any one of claims 18-19, wherein:
the multipotent ATZ stem cells do not express significant levels of CD45.

21. The method of any one of claims 18-20, wherein:
the multipotent ATZ stem cells express markers CD34, CD117 and CD184.

22. The method of any one of claims 18-21, wherein:
wherein the step of expanding the multipotent ATZ stem cells comprises replating the ATZ cells through at least 5 passages in MGM.

23. The method of claim 19, wherein:
the damaged or dysfunctional endodermal tissue is pancreatic tissue and the LSM comprises a pancreatic-specific LSM.

24. The method of claim 23, wherein:
wherein the LSM promotes differentiation of multipotent ATZ stem cells to produce a population of ATZ stem cell-derived pancreatic organoid cells.

25. The method of claim 19, wherein:
the damaged or dysfunctional endodermal tissue is hepatic tissue and the LSM comprises a hepatic-specific LSM.

26. The method of claim 25, wherein:
wherein the LSM promotes differentiation of multipotent ATZ stem cells to produce a population of ATZ stem cell-derived hepatic organoid cells.

27. The method of claim 19, wherein:
the damaged or dysfunctional endodermal tissue is lung tissue and the LSM comprises an lung-specific LSM.

28. The method of claim 27, wherein:
wherein the LSM promotes differentiation of multipotent ATZ stem cells to produce a population of ATZ stem cell-derived lung organoid cells.

29. The method of claim 19, wherein:
the damaged or dysfunctional endodermal tissue is intestinal mucosa tissue and the LSM comprises an intestinal mucosa-specific LSM.

30. The method of claim 29, wherein:
wherein the LSM promotes differentiation of multipotent ATZ stem cells to produce a population of ATZ stem cell-derived intestinal mucosa organoid cells.

31. A cellular composition, comprising:
a mixed population of ATZ cells comprising multipotent ATZ stem cells and endodermal progenitor ATZ cells in a pharmaceutically-acceptable carrier or exogenous biocompatible scaffold.

32. The cellular composition of claim 31, wherein:
the multipotent ATZ stem cells comprise at least 5% of the total ATZ cells.

33. The cellular composition of claim 31 or claim 32, wherein:
the multipotent ATZ stem cells do not express significant levels of CD45.

34. The cellular composition according to any of claims 31-33, for use as a medicament

35. A cellular composition for use in a method of treating damaged or dysfunctional endodermal tissue, in a subject, wherein:
the composition comprises isolated ATZ cells and/or ATZ-derived organoid cells.

36. The cellular composition for the use of claim 35, wherein:
the isolated ATZ cells and/or ATZ-organoid cells comprise ATZ crypt cells, such as ATZ multipotent stem cells.

37. The cellular composition for the use of claim 35 or claim 36, wherein:
the isolated ATZ cells and/or ATZ-organoid cells comprise endodermal progenitor ATZ cells.

38. The cellular composition for the use of any one of claims 35-37, comprising:
a pharmaceutically-acceptable carrier or exogenous biocompatible scaffold.

39. The cellular composition for the use of any of claims 35-38, wherein:
the subject is a human.

40. The cellular composition for the use of any of claims 35-39, wherein:
the damaged or dysfunctional tissue is gastrointestinal mucosa tissue.

41. The cellular composition for the use of any of claims 35-39, wherein:
the damaged or dysfunctional tissue is pancreas tissue.

42. The cellular composition for the use of any of claims 35-39, wherein:
the damaged or dysfunctional tissue is liver tissue.

43. The cellular composition for the use of any of claims 35-39, wherein:
the damaged or dysfunctional tissue is lung tissue.

44. The cellular composition for the use of any of claims 35-43, wherein:
the isolated ATZ cells and/or ATZ-derived organoid cells are allogenic to the subject.

45. The cellular composition for the use of any of claims 35-44, wherein:
the isolated ATZ cells and/or ATZ-derived organoid cells are autologous to the subject.
